(19) United States
(12) Patent Application Publication
(10) Pub. No.: US 2022/0138595 A1
(43) Pub. Date: May 5, 2022

(54) COMPUTER-BASED IDENTIFICATION OF TYPES IN THE EMPERICAL SCIENCES BY TANGLE THEORY

(71) Applicant: Universität Hamburg, Hanmburg (DE)

(72) Inventor: Reinhard Diestel, Hamburg (DE)

(21) Appl. No.: 17/648,243

(22) Filed: Jan. 18, 2022

Related U.S. Application Data
(63) Continuation of application No. PCT/EP2020/069402, filed on Jul. 9, 2020.

(30) Foreign Application Priority Data
Jul. 16, 2019 (DE) 10 2019 005 168.8

(51) Int. Cl. G06N 5/02; G16H 50/70

(52) U.S. Cl. CPC G06N 5/022; G16H 50/70

(57) ABSTRACT

A method of mechanically identifying and distinguishing clusters of qualities amongst potential qualities of given objects. Also a computer-readable storage device having stored thereon instructions for carrying out such method. Moreover, a system for mechanical exploitation of a data set containing relations of objects each with at least one respective quality, each object being an element of a given set of objects, the qualities each being included in a given list of potential qualities.

### RELATED APPLICATIONS

This application is a continuation of International Application No. PCT/EP2020/069402 filed on Jul. 9, 2020, which in turn claims priority to German patent application No. 10 2019 005 168.8, filed on Jul. 16, 2019, the entireties of both of which are hereby incorporated by reference.

The present invention concerns a method of mechanically identifying and distinguishing clusters of qualities amongst potential qualities of given objects. The invention further concerns a computer-readable storage device having stored thereon instructions for carrying out such method. Moreover, the invention concerns a system for mechanical exploitation of a data set containing relations of objects each with at least one respective quality, each object being an element of a given set of objects, the qualities each being included in a given list of potential qualities. The term ‘object’ in this document is used with its most general meaning; for example and in particular, objects can be things, concepts, creatures or persons, and so on.

Data sets such as those gained by surveys. experiments or other data gatherings often provide an accumulation of information that may contain unknown regularities, such as commonalities or similarities, whose identification may lead to new insights into the respectively considered objects and/or to improved possibilities of using the respective data set.

Different approaches, which may use computer-applied methods of artificial intelligence, are known to discover such regularities even in huge data sets. In particular, traditional clustering may be used to group objects that share certain qualities.

The present invention targets the problem of providing an alternative technique of automatically exploiting data sets.

The problem is solved by a. method, by a system and by a computer-readable storage device set out in the independent claims. Preferred embodiments are disclosed in the dependent claims and the description.

According to the present invention, the mathematical theory of tangles of abstract separation systems is used to identify, and distinguish, clusters of qualities amongst potential qualities (also referred to herein as ‘features’) of objects being elements of a given set.

Conversely to traditional clustering (which, as mentioned above, identifies groups of objects that share certain qualities), tangles identify groups of qualities that often occur together. They can thereby discover, relate, and structure types: of behavior, political views, texts, or viruses, for example.

Tangles offer a new, quantitative, paradigm for grouping phenomena rather than things. They can identify key phenomena that allow predictions of others. Tangles offer a new paradigm for clustering in large data sets.

The mathematical theory of tangles has its origins in the connectivity theory of graphs, which it has transformed over the past 30 years. It has recently been axiomatized in a way that makes it applicable to a wide range of contexts outside mathematics. This is explored here for the first time.

The text which follows explains the present invention as it illustrates. by discussing a range of simplified example scenarios, how tangles may be applied throughout the natural and social sciences: from clustering in data science to predicting customer behavior in economics, from DNA sequencing and drug development to text, analysis and machine learning.

Part 1. Introduction

1. The Idea Behind Tangles

This chapter offers three introductions to the concept and purpose of tangles. These introductions can be read independently, and readers may choose any one of them as an entry point to this document, according to their own background.

However as all three introductions illuminate the same concept, readers from any background are likely also to benefit from the other two viewpoints. Indeed, while each of them may seem plausible enough on its own, they are rather different. The fact that they nevertheless describe the same concept, that of a tangle, illustrates better than any abstract discussion the breadth of this concept and its potential applications, including in fields not even touched upon here. Moreover, even in a given context where one of the three viewpoints seems more fitting than the other two, switching to one of those deliberately for a moment is likely to add insight that would otherwise be easy to miss.

1.1 Tangles in the Natural Sciences

Suppose we are trying to establish the common cause of some set of similar phenomena. To facilitate this, we may design a series of measurements to test various different aspects of each of these phenomena.

If we already have an overview of all the potential causes, we might try to design these measurements so that every potential cause results in some expected reading for each measurement and different potential causes differ in at least one measurement. Then only the true cause would be compatible with all the readings we get from our measurements.

In our less-than-ideal world, it may not quite work like this. For a start, we might simply not be aware of all the potential causes—not to mention the fundamental issue of what, if anything, is a ‘cause’ Similar phenomena may have different causes. Our potential causes may not be mutually exclusive, in which case we will not be able to design experiments that will exclude all but one of them with certainty. And finally, measurements may be corrupted, but we may not know which ones were.

We usually try to compensate for this by building in some redundancy: perhaps by taking more measurements, or by measuring more different aspects. Or we might resign ourselves to making claims only in probability—which will protect us from being disproved by any single event, but which may also increase immensely the overheads needed to justify precise quantitative assertions (of probabilities).

Tangles offer a structural, rather than probabilistic, way to afford the redundancy needed in such cases, to do so in a particularly economical way, and to sidestep the philosophical issue of what constitutes a cause

In our example, a tangle would be a hypothetical collection of readings for all the measurements taken, a set of one possible reading per measurement. It would not he just any such collection, but one that is typical for the actual collections of readings we got from the phenomena we measured, in one of two ways to be described in a moment.¹ It may happen that one, or several, of our phenomena produced exactly this set of readings, but it can also happen that an ‘abstract’ set of readings is typical, and hence a tangle, for our collection of phenomena without occurring exactly in any one of them. ¹ Recall that we performed the same measurements on each of the phenomena we are investigating, so we have one set of readings for each phenomenon.

Our measurements might yield a single tangle, or several, or none. Given any one of them, we may try to find a common cause for this typical set of readings, or choose not to try. If there is a common cause for many of the phenomena investigated, it will show up as a tangle and can thus be identified.

But there can also be tangles that cannot—or not yet—be ‘explained’ by a common cause. Such tangles are just as substantial, and potentially useful, as those that can be labelled by a known common cause; indeed perhaps more so, since the absence of an obvious common cause may have left them unidentified in the past. In this sense, identifying tangles in large sets of phenomena can lead to the discovery of new meta-phenomena that had previously gone unnoticed and might, henceforth, be interpreted as a ‘cause’ for the group of phenomena that gave rise to this tangle.

So when is a set τ of hypothetical measurement readings deemed ‘typical’ for the actual measurements of our phenomena, and therefore a tangle? There are two notions of ‘typical’ that are important in tangle theory: a strong one that is satisfied by most tangles but not required in their definition, and a weaker one that is required in their definition, and which suffices to establish the main theorems about tangles.

The strong notion, which we might call popularity-based, is that our set of phenomena has a subset X (not too small) such that, for every measurement taken, at least 80% of the phenomena in X give the reading laid down in τ.² Note that these will be different sets of 80% of X for different measurements: every phenomenon, even in X, may for some measurements produce a reading different from the reading that t prescribes for that measurement. Clearly, there can be several such tangles τ, witnessed by different sets X of phenomena. ² Thus, our fixed abstract collection τ of hypothetical measurement readings is ‘popular’ with the elements of X.

The weaker notion of when our set τ of readings is ‘typical’, and hence constitutes a tangle, might be called consistency-based. It says that for every small set of up to three measurements there should be a few phenomena, at least n, say, that gave the reading specified by τ for these three measurements. In particular, no subset σ of up to three elements of τ proves τ to be ‘inconsistent’ in that none of the phenomena we investigated produced exactly the readings in σ.³ Note that if τ is typical in the popularity-based sense it will also be typical in the consistency-based sense⁴, but not conversely. ³ Three readings that are inconsistent in the usual sense that they cannot occur together, because they contradict each other, would be an example. But since the theory of tangles is, and should be, independent of interpretation, we cannot take recourse to logic and have to work with the concrete set of phenomena at hand. The reason why we work with subsets of size up to three, rather than two, may be surprising but is immaterial at this informal level.⁴ . . . as long as X is large enough that 4/10|X|≥n. Indeed, let σ consist of the results of the measurements A, B, C laid down in τ. For each of A, B, C at most 20% of the phenomena in X disagree with τ, so at least 100-60=40 percent of X agree with τ on all of A, B and C—which is at least n phenomena, as required.

Note that both these notions of ‘typical’ are robust against small changes in our set of data. This makes tangles well suited to ‘fuzzy’ data with the kind of imperfections indicated earlier.

1.2. Tangles in the Social Sciences

Suppose we run a survey S of fifty political questions on a population P of a thousand people. If there exists a group of, say, a hundred like-minded people among these, there will be a ‘typical’ way of answering the questions in S in the way most of those people would. Quantitatively, there will exist a subset X of P, not too small, and an assignment τ of answers to all the questions in S such that, for most questions sϵS, some 80% (say) of the people in X agree with the answer to s given by τ. (Which 80% of X these are will depend on the choice of s.) We call this collection τ of views—answers to the questions in S—a mindset. Note that there may be more than one mindset for S, or none.

Traditionally, mindsets are found just intuitively: they are first guessed, and only then established by quantitative evidence from surveys designed to test them. For example, we might feel that there is a ‘socialist’ way 6 of answering S. To support this intuition. we might then check whether any sizable subset X⊆P as above exists for this particular τ=σ.

Tangles can do the converse: they will identify both X and τ without us having to guess them first:

Tangles offer a precise, quantitative, way to identify known mindsets and to discover unknown ones.

For example, tangle analysis of political polls in the UK in the years well before the Brexit referendum might have established the existence of a mindset we might now, with hindsight, call the ‘Labour-supporting non-socialist Brexiteer’: a mindset whose existence few would have guessed intuitively when Brexit was not yet on the agenda. And similarly in the US with the MAGA⁵ mindset before 2016, or that of a ‘conservative Green’ in the early 1970s. Tangles can identify previously unknown patterns of coherent views or behavior. ⁵ Make America Great Again; Donald Trump's 2016 presidential campaign slogan.

1.3 Tangles in Data Science

One of the most basic, and at the same time most elusive, tasks in the analysis of big data sets is clustering: given a large set of points in some space, one seeks to divide the set into a small number of subsets, called ‘clusters⋅; of points that are in some sense similar Similarity is usually defined in terms of a distance function on the pairs of points, so that sets are pairwise close become a cluster.

The concept is explained with regard to the accompanying drawings. These show

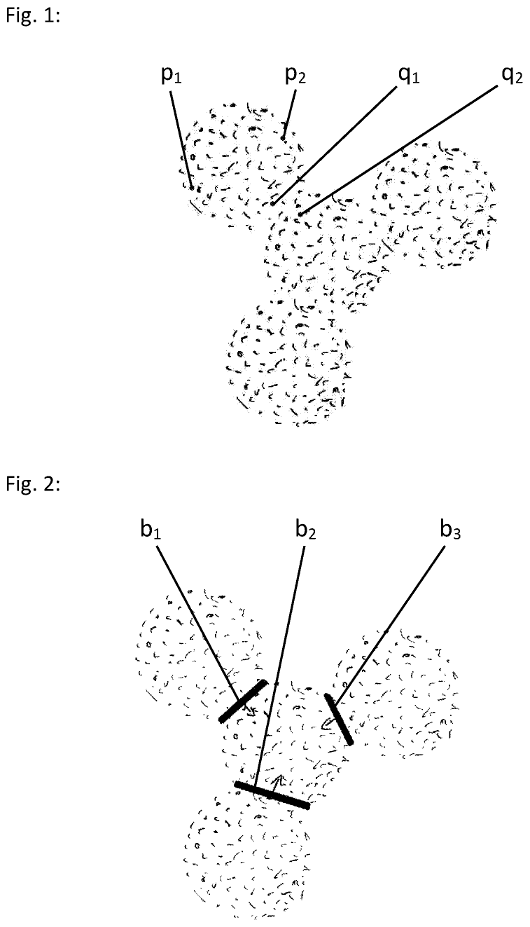

FIG. 1: four presumptive clusters;

FIG. 2: the point set of FIG. 1 with a consistent orientation of bottlenecks towards the central cluster; and

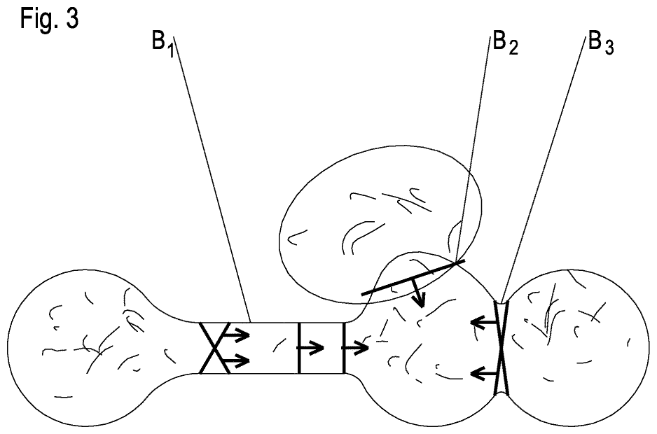

FIG. 3: a point set with three bottlenecks, but many bottleneck partitions; therein, the central cluster orients them all consistently.

FIG. 1 shows a simple example of points in the plane. We can clearly see four clusters. Or can we? If a cluster is a set of pairwise close points, and the two points p1, p2 lie in the same cluster, should not the two points q1, q2 which are much closer lie in a common cluster too?

For reasons such as this, and other more subtle ones, there is no universal notion of when two points in a data set are deemed to be ‘close’. And even if there is a consensus in a particular clustering application about such a distance function, there are still many ways of defining clusters based on this metric—even for such a simple setting as points in the plane.

Tangles define clusters in an entirely different manner. Not by dividing the data set in some clever new way, but without dividing it up at all: although there will be four tangles in our picture, these will not be defined as sets of points. In particular, questions such as whether the points p1, p2 should end up in the same cluster but the points q1, q2, perhaps, should not, do not even arise.

By avoiding the issue of assigning points to clusters altogether, tangles can be precise without making arbitrary and unwarranted choices:

Tangles offer a precise, but robust, way to identify fuzzy clusters.

Rather than looking for dense clouds of data points, tangles look for the converse: for obvious ‘bottlenecks’ at which the data set naturally splits in half—or more precisely, into two subsets, no matter how large or small. We call ways of splitting our data set into two disjoint subsets partitions of the set, and the two subsets the sides of the partition.

FIG. 2 shows three partitions of our point set at bottlenecks b₁, b₂ and b₃.⁶ Now, whatever formal definition of cluster (and of bottleneck) we might choose to work with, one thing will be clear: no bottleneck partition will divide any cluster roughly in half, since that would violate either our definition of a cluster of that of a bottleneck. For example, given one of the three bottlenecks in our picture, and one of the four obvious clusters, we might argue over a few points about whether they should count as belonging to that cluster or not, and if so on which side of the bottleneck they lie. But for almost all the points in our picture these questions will have a clear answer once we consider a fixed cluster and a fixed bottleneck, no matter how loosely defined. ⁶ The partition of our set at the bottleneck b₂, for example, has the bottom cluster on one side and the other three clusters on the other side.

Put another way, whichever precise definitions we were to choose, each of our four clusters would lie mostly on the same side of any partition at a bottleneck. Let us then say that the cluster orients this partition towards the side on which most of it lies. FIG. 2 shows how the central cluster, no matter how we choose to define it precisely, orients the partitions at the three bottlenecks in this way. Each of the four clusters assigns its own set of arrows to these same three partitions, and the central cluster orients them all inwards.

Note that assignments of arrows (to partitions at bottlenecks) that come from one of the four clusters in this way are not arbitrary: the arrows are ‘consistent’ in that they all point roughly in the same way, namely, towards that cluster.

The key idea behind tangles, now, is to keep for each cluster exactly this information—how it orients all the bottleneck partitions—and to forget everything else (such as which points belong to it). More precisely, tangles will be defined as such abstract objects: as ‘consistent orientations of all the bottleneck partitions’ in a data set. In this way, tangles will extract from the various explicit ways of defining clusters as point sets something like their common essence. Tangles will be robust against small changes in the data, just as they are robust against small changes in any explicit definition of point clusters we might use to specify them. But their definition as such will be perfectly precise, and involve no arbitrary choices of the kind one invariably has to make when one tries to define point clusters as sets of points.

Of course, given a data set one has to define formally what its bottleneck partitions are, and when an orientation of all the bottleneck partitions is deemed to be ‘consistent’.⁷ The challenge is to do all this without reference to any perceived cluster, however vaguely defined: we can only define clusters indirectly as tangles, as is our aim, if our definition of a tangle and in particular our definition of consistency—does not itself refer to explicit clusters given as point sets.⁸ ⁷ For example, orienting the three bottleneck partitions in FIG. 2 outwards should he ‘inconsistent’, as the arrows would point away from each other.⁸ In discussing our example, so far, we did the converse: we assumed we knew roughly what the clusters should be, we thought of a bottleneck as a place at which we could partition our big set without cutting right through any big cluster, and we defined the consistency of arrows at bottlenecks by reference to those clusters, as pointing towards them. This was fine for the purpose of motivating the concept, of tangles: after all, in cases where we feel we know roughly what the clusters should be, tangles should identify precisely these. But once we get serious about defining tangles formally, we must no longer appeal to pictures or intuitive examples.

To make the problem a little clearer, let us look at a slightly modified example. FIG. 3 again shows four clusters and three bottlenecks B₁, B₂ and B₃. But this time one of the bottlenecks has an elongated shape, like a handle. The points in this handle do not really belong to any cluster—or, if we insist on assigning every point to some cluster, there are two clusters to which they might be assigned, with equally reasonable justification. The example further illustrates that, even for reasonable definitions of ‘bottleneck’ (which should include the handle) there may well be several partitions ‘at’ the same bottleneck.⁹ ⁹ This is the same problem in disguise, that of assigning points to sets: since a partition is a pair of sets, changing these sets even slightly will give us a different partition. Ideally, we would like there to be only one partition at each bottleneck. But since there is no canonical way to choose one from the many candidates, we have to admit them all at this stage.

Now, if clusters are going to be tangles, and tangles are to be consistent orientations of all the bottleneck partitions, then our intuition that we want there to be only four clusters in FIG. 3 dictates that only four of the many ways of orienting all its bottleneck partitions—one for every desired cluster—should count as ‘consistent’. In the picture this can be achieved if, and only if, we can ensure that consistent orientations of partitions at the same bottleneck always point the same way. Indeed, in this case consistent orientations of the (many) bottleneck partitions will induce orientations of the (few) bottlenecks themselves, which intuitively is our aim.

In our example, the orientations of bottleneck partitions induced by one of the four ‘obvious’ clusters satisfy this nicely: any given cluster will either lie mostly on the left of every partition at the handle, or mostly on the right of every partition at the handle. Hence, the arrows defined at these partitions by any of our four clusters will either all point to the left, or they will all point to the right, and thus be intuitively consistent.

The challenge remains to come up with a formal definition of consistency as the basis for our notion of tangle that bears this out: one that does not refer to any perceived clusters, but which in the above example will orient all the partitions at the handle in the same direction. Chapter 2 shows how this can be done.¹⁰ 10 The term ‘consistency’ will be given a slightly narrower meaning there than in the present discussion. But tangles will be orientations of all the ‘bottleneck’ partitions that are consistent in our stronger sense here; such orientations will be called ‘typical’ in Chapter 2.

Once that is achieved, we shall have a definition of tangle which, while being entirely formal and precise, will be able to capture ‘fuzzy’ clusters in a robust way that does not require us to allocate points to clusters.

2. The Notion of a Tangle

Consider a collection V of objects and a set \vec{S} of features (also referred to herein as ‘qualities’)¹¹ that each of the objects in V may have or fail to have. Given such a (potential) feature \vec{s}ϵ\vec{S}, we denote its negation by [symbol] . The pair {s, [symbol] } of the feature together with its negation is then denoted by s, and the set of all these s is denoted by S. 11 Logicians may prefer to say ‘predicates’ instead of ‘features’ here. That would be correct, but I am trying to avoid any (false) impression of formal precision at this stage.

For example, if V is a set of pieces of furniture, then \vec{s} might be the feature of being made of wood. Then [symbol] would be the feature of being made of any other material, or a combination of materials, and s could be thought of as the question of whether or not a given element of V is made of wood.

In the language of Chapter 1.1 the elements of V would be the phenomena investigated. The sϵS would be the measurements performed on these phenomena, with two possible outcomes \vec{s} and [symbol] (called ‘readings’ in Chapter 1.1).

In the example of Chapter 1.2, the set V would be the population P of people polled by our survey S. which for simplicity we assume to consist of yes/no questions. Then {\vec{s}|sϵS} might be the set of “yes” answers to the questions in S while [symbol] would denote the “no” answer to the question s.

In the clustering scenario of Chapter 1.3. the set V would be the set of points in which we look for clusters. If we equate a feature s with the set of objects, in V that have it, then \vec{s} and [symbol] form a partition of V, the partition s={\vec{s}, [symbol] }. We may think of S as the set of those partitions of V that are particularly natural, its ‘bottleneck’ partitions.

2.1 Features that Often Occur Together

Tangles are a way to formalize the notion that some features typically occur together. They offer a formal way of identifying such groups of ‘typical’ features, each ‘type’ giving rise to a separate tangle.

In order to identify a collection of features as ‘typical’, it is not necessary to precisely delineate a corresponding set of objects (elements of V) that have precisely, or even mostly, these features. This reflects most real-world examples, where these sets are at best ‘fuzzy’. By working directly on the level of features rather than the level of objects, tangles can be completely precise even when the objects whose features they capture cannot be clearly delineated from each other. This is a particular strength of tangle theory compared with traditional clustering methods.

Let us return to the example where Vis a set of pieces of furniture. Our list \vec{S} of possible features (including their negations) consists of qualities such as color, material, the number of legs. intended function, and so on—perhaps a hundred or so potential features. The idea of tangles is that, even though \vec{S} may be quite large, its elements may combine into groups that correspond to just a few types of furniture as we know them: chairs, tables, beds and so on.

The important thing is that tangles can identify such types without any prior intuition: if we are told that a container V full of furniture is waiting for us at customs in the harbor, and all we have is a list of items v identified only by numbers together with, for each number, a list of which of our 100 features this item has, our computer—if it knows tangles—may be able to tell us that our delivery contains furniture of just a few types: types that we (but not our computer) might identify as chairs, tables and beds, perhaps with the tables splitting into dining tables and desks.

In the language of Chapter 1.1, these types would correlate with the different possible ‘causes’ for objects to be furniture: our need to sit, sleep, use computers and so on. In the example of Chapter 1.2, they would be mindsets. In the setting of Chapter 1.3, the sets of chairs, tables and beds would form clusters in V. These clusters might not be clearly delineated—for example, if our delivery contains a deckchair—but the types, groups of features that often occur together, would be precisely defined.

In the remainder of this chapter we shall not always make explicit reference to the three example scenarios from Chapter 1. But readers are encouraged to check for themselves what the various new terms mean in each of those contexts, to keep all three aspects alive as they build their intuition for tangles.

2.2 Consistency of Features

To illustrate how our computer may be able to identify types of furniture from those features lists without understanding them, let us briefly consider the inverse question: starting from a known type of furniture, such as chairs, how might this type be identifiable from the data if it was not known?

A possible answer, which will lead straight to the concept of tangles, is as follows. Each individual piece of furniture in our unknown delivery, vϵV say, has some of the features from our list \vec{S} but not others. It thereby specifies the elements s of S: as \vec{s} if it has the feature \vec{s}, and as [symbol] otherwise. We say that every vϵV defines a specification of S, a choice for each sϵS of either \vec{s} or [symbol] but not both. We shall denote this specification of S as

$$v ( S ) : = \{ v ( s ) \vert s ϵ S \} ,$$

where v(s):=\vec{s} if v specifies \vec{s} as \vec{s} and v(s):= [symbol] if v specifies s as [symbol] .

Conversely, does every specification of S come from some vϵV in this way? Certainly not: there will be no object in our delivery that is both made entirely of wood and also made entirely of steel. Thus no vϵV will specify both r as \vec{r} rather than [symbol] , and s as \vec{s} rather than [symbol] , when \vec{r} and \vec{s} stand for being made of wood or steel, respectively. In plain language: no specification of S that comes from a real piece of furniture can contain both \vec{r} and \vec{s}, because these features are inconsistent.

Let us turn this manifestation in V of logical inconsistencies within \vec{S} into a definition of ‘factual’ inconsistency for specifications of S in terms of V. Let us call a specification of S consistent if it contains no inconsistent triple, where an inconsistent triple is a set of up to three¹² features that are not found together in any vϵV. Specifications of S that come from some vϵV are clearly consistent. But S can have many consistent specifications that are not, as a whole, witnessed by any vϵV.¹³ ¹² It might seem more natural to say ‘two’ here, as in our wood/steel dichotomy above. Our definition of consistency is a little more stringent, because the mathematics behind tangles requires it. Note that, formally, the elements of an inconsistent ‘triple’ need not be distinct; an ‘inconsistent pair’ of two features \vec{r}, \vec{s} not shared by any vϵV, for example, also counts as an ‘inconsistent triple’, the triple {\vec{r}, \vec{r}, \vec{s}}={r, s}.¹³ Here is a simple example. Suppose some of our furniture is made of wood, some of steel, some of wicker, and some of plastic. Denote these features as \vec{p}, q, \vec{r}, s, respectively, and assume that S={p, q, r, s}. Then the specification τ={ [symbol] , [symbol] , [symbol] , [symbol] } is consistent, because for any three of its elements there are some items in V that have none of the three corresponding features: those that have the fourth. But no item fails to have all four of these features. So the consistent specification τ of S does not come from any one vϵV. We shall get back to this example in Chapter 6.5.

Tangles will be specifications of S with certain properties that make them ‘typical’ for V. Consistency will be a minimum requirement for this. But since any specification of S that comes from just a single vϵV is already consistent, tangles will have to satisfy more than consistency to qualify as ‘typical’ for V.

2.3 from Consistency to Tangles

It is one of the fortes of tangles that they allow considerable freedom in the definition of what makes a specification of S ‘typical’ for V—freedom that can be used to tailor tangles precisely to the intended application. We shall describe this formally in Chapter 6. But, we are already in a position to mention one of the most common ways of defining ‘typical’, which is just a strengthening of consistency.

To get a prior feel for our (forthcoming) formal definition of ‘typical’, consider the specification of S in our furniture example that is determined by an ‘ideal chair’ plucked straight from the Platonic heaven: let us specify each sϵS as \vec{s} if this imagined ideal chair has the feature \vec{s}, and as [symbol] if not. This can be done independently of our delivery V, just from our intuitive notion of what chairs are. But if our delivery has a sizable portion of chairs in it, then this phantom specification of S that describes our ideal chair has something to do with V after all.

Indeed, for every triple \vec{r}, \vec{s}, \vec{t} of features of our ideal chair there will be a few elements of V, at least n say, that share these three features. For example, if \vec{r}, \vec{s}, \vec{t} stand for having four legs, a flat central surface, and a near-vertical surface, respectively, there will be—among the many chairs in V which we assume to exist—a few that have four legs and a flat seating surface and a nearly vertical back.

By contrast, if we pick twenty rather than three features of our ideal chair there may be no vϵV that has all of those, even though there are plenty of chairs in V. But for every choice of three features there will be several—though which these are will depend on which three features of our ideal chair we have in mind

Simple though it may seem, it turns out that for most furniture deliveries and reasonable lists S of potential features this formal criterion for ‘typical’ distinguishes those specifications of S that describe genuine types of furniture from most of its other specifications.¹⁵ But in identifying such specifications as ‘types’ we made no appeal to our intuition, or to the meaning of their features.¹⁶ ¹⁵ . . . of which there are many: if S has 100 elements, there are 216¹⁰⁰ specifications of S.¹⁶ This is not to say that the use of tangles is free of all preconceptions, biases etc. For example, the choice of a survey S in the scenario from Chapter 1.2 is as loaded or neutral as is would be in any other study that starts with a survey. The statement above is meant relative to the given S once chosen. In Chapter 6 we shall discuss how the deliberate use of preconceptions, e.g. by declaring some questions in S as more fundamental than others, can help to improve tangles based on such preconceptions. We shall also see how to do the opposite: how to find tangles that arise naturally from the raw data of S and V, without any further interference from ourselves.

So let us make this property of specifications of S that describe ‘ideal’ chairs, tables or beds into our formal, if still ad-hoc, definition of ‘typical’: let us call a specification τ of S typical for V if for every set R of at most three elements of S there are at least n elements v of V that specify R as τ does, i.e., for which v(R)=τ(R). (The integer n here is a fixed parameter on which our notion of ‘typical’ depends and which we are free to choose.)

Crucially, this definition of ‘typical’ is purely intrinsic: it depends on V, but it makes no reference to what a typical specification S ‘is typical of’. Specifications of ideal chairs, tables or beds are all typical in this sense: they all satisfy the same one definition.

Equally crucially, a specification of S can be typical for V even if V has no element that has all its features at once. Thus, we have a valid and meaningful formal definition of an ‘ideal something’ even when such a thing does not exist in the real world, let alone in V.

Relative to the definition of ‘typical’ we can now define tangles informally:

A tangle of S is any specification of S that is typical for V.

Since our ad-hoc definition of ⋅’typical’ is phrased in terms of small subsets of \vec{S}, sets of size at most 3 (of which there are not so many), we can compute tangles without having to guess them first. In particular, we can compute tangles of S even when V is ‘known’ only in the mechanical sense of data being available (but not necessarily understood), and S is a set of potential features that are known, or assumed, to be relevant but whose relationships to each other are unknown.

Tangles therefore enable us to find even previously unknown ‘types’ in the data to be analyzed: combinations of features that occur together significantly more often than others. This was important in all three of the scenarios from Chapter 1: tangles can identify previously unknown causes, mindsets, or clusters.

2.4 Witnessing Sets and Functions

When we just defined a tangle of S as any specification τ of S that is typical for V, we were assuming a notion of ‘typical’ that we called consistency-based in Chapter 1.1: for every set R of up to three elements of S there should be at least n elements of V that specify R as τ does, for some fixed integer n. This notion of a tangle will form the basis for the angle theory developed later.

In Chapter 1.1 we also discussed another possible notion of ‘typical’, which we called popularity-based. This was that V has a subset X, not too small, in which τ is ‘popular’ in that for every sϵS some 80% of the elements of X specify s as τ does. We saw that, if X is big enough relative to n, then this implies that τ is typical also in the earlier sense, and hence is a tangle. We may thus think of X as ‘witnessing’ this.

In our furniture example, the tangle of being a chair will be witnessed by the set X of chairs in V. every individual feature of our ‘ideal chair’ τ will be shared by some 80% of all the chairs in V, though not all by the same 80%. Such witnessing sets were also used in Chapter 1.2, where we defined a mindset as a collection of views established by a political survey S that where ‘often held together’, in exactly this sense.

Formally, let us say that a set X⊆V witnesses a specification τ of S if, for every sϵS, there are more v in X that specify s as τ does than there are vϵX that specify s in the opposite way. If these majorities are greater than ⅔, then τ will be a tangle as defined in Section 2.3, at least for n=1, no matter how large or small X is.

More generally, let us say that a ‘weight’ function w: V→N witnesses τ if, for every sϵS, the collective weight of the vϵV that specify s as τ does exceeds the collective weight of the vϵV that specify s in the opposite way.¹⁷ ¹⁷ Formally, w: V→N witnesses τ if Σ{w(v)|v(s)=τ(s)}>Σ{w(v)|v(s)≠τ(s)} for every sϵS.

Much of the attraction and usefulness of tangles stems from the fact that, in practice, most of them have such witnessing sets or functions [3]. But it is important to remember that the definition of a tangle, be it our preliminary definition from Section 2.3 or the formal one given later, does not require that such sets or functions exist. It relies only on notions of consistency and of type, which are both defined by banning triples in \vec{S} deemed ‘inconsistent’ or ‘atypical’ from occurring together in a tangle. So far, both these were defined with reference to the values of v(S) for vϵV, and being typical was simply a strengthening of consistency.

In some contexts, however, tangles of S can be defined without any reference to V at all. In our furniture example we could have defined the consistency of a set of features, or predicates, about the elements of V in purely logical or linguistic terms that make no appeal to V. Indeed if \vec{r} stands for ‘made entirely of wood’ and \vec{s} stands for ‘made entirely of steel’, then the set {\vec{r}, \vec{s}} is inconsistent. The reason we chose to give was that no object in V is made entirely of wood and also made entirely of steel. But we might have said instead that these two predicates are logically inconsistent—which implies that there is no such object in V but which can be established without examining V.

The way consistency and type are defined formally [2] as part of the notion of abstract tangles is something half-way between these two options: it makes no reference to V but refers only to some axiomatic properties of \vec{S} which reflect our notion that \vec{S} is a set of ‘features’. In this way it also avoids any appeal to logic or meaning.

For the rest of this document the only important thing to note about witnessing sets or functions is that while many tangles have them, tangles can be identified, distinguished, or ruled out without any reference to such sets or functions. The mindset of being socialist can be identified without having to find any actual socialists, let alone delineating these as a social group against others.

Part II: Tangles in Different Contexts: A Collection of Informal Examples

The aim of the three chapters in this Part II of the document is to indicate the range of potential uses of tangles by a diverse collection of simple, synthetic, examples from different contexts. Our aim will be to flush out the notion of a tangle, as developed in Part I, by viewing it in these contexts, not (yet) to discuss what applying tangles can achieve there.

Since I am not an expert in any of the disciplines from which the examples described in the next three chapters are taken, I shall not attempt, more than to indicate the kind of use that tangles might find there: any actual use will require genuine expertise in the respective field. However, the examples discussed here may serve as templates for such an endeavor: they should be diverse enough to suit most contexts, their description simple enough to be inspiring.

The default format for describing our examples will be to . . .
name the set V of objects studied;
describe the set S of their potential features:
discuss when a specification of S is deemed to be typical:
see what tangles—typical specifications—of S mean in this context.

In Chapter 2.3 we defined a tangle of S as a specification τ of S that, is ‘typical’ for V. Let us now formalize a little more what this shall mean from now on. For a start, τ has to be consistent: every three of its features must be found combined in at least one element of V.¹⁸ A roundabout way of saying the same thing is that τ must not contain an inconsistent triple: a set of at most three features not found together in any element of V. ¹⁸ In terms of the notation introduced in Chapter 2.2: τ is consistent if for every set R of at most three elements of S there exists some vϵV such that v(R)=τ(R).

Sometimes, consistency is enough for us to think of a specification of S as typical for V; then the tangles of S are precisely its consistent specifications. At other times we wish to demand a little more of a tangle. We shall do that by declaring some further small sets of elements of \vec{S} as ‘atypical’, and then calling a specification of S typical if it is consistent and contains none of these ‘forbidden’ subsets. The collection of these ‘forbidden’ atypical sets will usually be denoted by [symbol] .¹⁹ ¹⁹ ln our earlier case, where we wanted all consistent specifications of S to count as tangles, we simply choose [symbol] to be the empty set to avoid placing any further restrictions on τ.

ln Chapter 2.3 we implicitly chose as [symbol] the set [symbol] , of triples of features such that fewer than n elements of V have these particular (up to three) features, where n is a parameter we are free to choose. Large values of n make it harder for a specification of S to count as ‘typical’, choosing n small makes it easier.

With this understanding, tangles continue to be typical specifications of S: consistent sets of features that contain exactly one of \vec{s} and [symbol] for every sϵS and have no subset in [symbol] .

If we work with [symbol] = [symbol] ₙ₁, the choice of n will be our main tool to influence how many tangles S has: high values of n will give us fewer more pronounced tangles, while lower values will give us more but less valuable tangles.²⁰ ²⁰ In Chapter 6.5 we shall meet another tool that lets us influence the number of tangles found, so-called order functions on tangles. That tool works by placing restrictions—strong or not so strong—on S itself. It influences not only the number of tangles, in more subtle ways than the choice of n for [symbol] ₙ, but also how they relate to each other.

3. Examples from the Natural Sciences

This chapter is written using the informal notation from Chapter 2. We have a set V of ‘objects’, and a set \vec{S} of ‘features’ of these objects. These features come in pairs s={\vec{s}, [symbol] }, which we call ‘potential features’. The features \vec{s} and [symbol] are the two specifications of s. Every object vϵV has one of the features \vec{s} or [symbol] for every sϵS, but never both: We denote the one it has by v(s) and call it v's specification of s.

The potential features s typically correspond to measurements that can be applied to the objects in V, with \vec{s} and [symbol] as the two possible outcomes.²¹ ²¹ Measurements with more than two, but finitely many, outcomes can be modelled as several measurements of this simple 0/1 kind. We shall discuss this later in more detail.

A specification of S is a choice of either \vec{s} or [symbol] for each sϵS. It is consistent if for every three of its choices, \vec{r}, \vec{s}, \vec{t}, say, there exists an object vϵV that specifies r, s and tin exactly this way, as v(r)=\vec{r}. A consistent specification of S is typical for V if it has no subset in some collection [symbol] of subsets of \vec{S} which we shall specify in every context.

A tangle of S is a typical specification of S, one that is consistent and has no subset in [symbol] .

3.1 Expert Systems: What Exactly is an Illness?

What exactly is an illness? Since the advent of modern medicine. we have come to think of an illness in terms of its cause: a common cold, for example, is an infection by a certain type of virus. However, notions of illnesses usually predate the discovery of their cause. In those cases, illnesses are defined in terms of what we see, no matter whether we understand it: as collections of symptoms. Such definitions, by nature, are much vaguer. As medical research advances, shall we seek to abandon these symptomatic definitions of illnesses in favor of naming one, or perhaps a definitive combination of a few, measurable conditions whose exact presence defines an illness? Or shall stick with the symptomatic definitions—and if so, is there anything we can do to handle their intrinsic vagueness in precise, quantifiable, and intersubjective ways?

Even from a modern perspective, illnesses do not normally have a single cause. This might still be argued in rare cases, such as a missing gene. But even an infection by a virus that is often behind the common cold turns into an illness only if other ‘causes’ are present too, such as a weakened immune system. And besides, even if a single cause, or definitive set of causes, exists, this will rarely be what we see: what we see is a patient who feels unwell. Such a patient will come with some obvious symptoms and can be tested for some less obvious ones. Doing this before making a wild guess at a single cause is a time-tested, and still entirely sensible, process of diagnosis.

Doctors like to speak of individual tests in this process as ‘excluding’ some potential cause. The idea is that any given hypothetical cause of the illness would also cause certain measurable others conditions, such as body temperature in a certain range, so if the measured temperature lies outside this range then this hypothetical cause cannot be the cause of the illness. As we all know, while this makes sense in principle, it is unlikely to work in every case: humans are just too complex, and the condition whose measurement we hope might exclude our potential cause is likely also influenced by a host of other parameters beyond our control.²² ²² It may work better in controlled environments, such as diagnosing the cause for a computer crash.

What doctors will do in such cases is work with intuitive probabilities: while each symptom tested my sometimes fail to be present as expected, the chance that this fails for many independent symptoms is much smaller, and so the diagnostic conclusion drawn (such as the exclusion of a certain cause) becomes more certain. An expert system should then advise them on just what the probabilities are—pretty daunting task considering the number of experiments it takes to make probabilistic statements with a given degree of confidence.

Tangles can cope with the intrinsic fuzziness in how illnesses are reflected by symptoms in a structural, rather than probabilistic, way.²³ They can serve to identify illnesses from sets of diagnostic measurements, and they can help identify combinations of diagnostic results as ‘typical’—in which case these could, and maybe should, be thought of as a previously unknown illness that doctors may wish to be aware of in the future. ²³ Although medical diagnoses may be a particularly fitting example, the approach can be used in other diagnostic contexts too, such as for machine failure.

Let us now have a look at how this might work in the setting of Chapter 2: how illnesses correspond to tangles. The first of the main two tangle theorems, applied to these tangles, will find what in our context amounts to an expert system for the diagnostic process of identifying an illness: a small set of measurements which, between them, suffice to distinguish different illnesses encoded as tangles with maximum redundancy (Chapter 8.1). Once a particular tangle has been identified by the system, it, can then be verified further by additional measurements of the symptoms in terms of which it is defined.

So how can an illness be described as a tangle? In our model, V might be the set of all patients on record. The set S might consist of measurements, ideally (for simplification) with just two potentials results: s for a ‘positive’ reading indicating an abnormality (such as ‘increased pulse’), and [symbol] for the corresponding ‘negative’ reading (‘pulse slow or normal’).²⁴ ²⁴ So testing for an abnormally slow pulse would be considered as another measurement. Similarly, there could be one measurement testing for body temperature in the range typical for viral infections, and another for bacterial infections, although these can be taken together in the same physical measurement of body temperature.

Any illness then defines a specification of S, specifying those measurements s E S as \vec{s} that usually, though not necessarily, have a positive reading when the illness is present, and all other s as \vec{s}. Such a specification τ of S will be ‘typical’ in both senses discussed in Chapter 2, and will hence be a tangle.²⁵ ²⁵ More precisely, we require of S that if a positive reading s is associated with an illness then more than two thirds—but by no means all—of the patients with this illness have this positive reading \vec{s}, and that for all other measurements in S we have fewer than a third false positives. Then the set X of patients having the illness will witness τ in the sense of Chapter 2.4, making it a tangle at least, for [symbol] =0.

Conversely, every tangle of S is a collection of symptoms that often occur together. This alone justifies giving this collection of symptoms some attention, even if it is not normally associated with any known common illness.

3.2 Text Tangles: Identifying Topic, Genre, Authors

Training computers to compare texts is a well-established topic in computer science. A number of methods have been suggested that use various criteria to gauge the similarity of two given texts. ranging from word counts to syntax comparisons. When these “metrics” are used for standard clustering algorithms, they will find corresponding clusters in a given set V of texts: subsets of texts that are more similar to each other than to the other texts in V.

The same criteria can be used to define ‘features’ of the elements of V, among which we can then look for tangles. For example, the particularly frequent use of a certain word, or of a grammatical construction, could be such a feature. A tangle, then, will be a particular collection of features typical for some of the texts in V. In other words, tangles will be types of text, not sets of (similar) texts themselves.

The set S of potential features to look for will depend on our collection of texts: features of syntax that help us compare novels, or novelists, may be less useful when we analyze computer manuals, and word counts for flowers may help with identifying gardening books amongst texts about hobbies but not when we try to sort legal texts. If we already know something about the texts in V, or if we are interested in finding types of a particular kind, we may wish to design S accordingly.

But meaningful collections S of potential features can also be computed mechanically. For example, we might identify some words that occur considerably more frequently than average, either in a particular text or in the texts in V as a whole. For such words we could create a potential feature s, making \vec{s} the feature that this word does occur particularly often.

With S chosen, or computed, in this way, one might expect its tangles to capture types of text that can be defined in terms of these features. Thus, if many of our features correspond to the frequent use of particular words, we would expect tangles of S to reflect the topic of a text. If they are chosen to correspond to particular syntactic patterns, they might reflect the text's genre. And if our pre-processing is used to identify phrases used particularly often, they might even correspond to authors.

Interestingly, while this does seem to work in principle, it does not quite work with the kind of S outlined above. This will be a topic for Chapter 8.2, but we can already see the problem now. Let us indicate it by a simple example.

Suppose V is a collection of texts about hobbies. Our hope is that tangles can identify these hobbies as types of text—including, perhaps, some surprises that identify combinations of interests as hobbies that no-one had thought of. If there are enough texts about gardening in our collection V, we would expect ‘gardening’ to appear as a tangle. Formally, this tangle would be a ‘typical’ specification of S, which would include the use of words such as—‘rose’, ‘lawn’ or ‘watering’ as features g and words such as ‘yeast’, ‘flour’ or ‘pre-heated’ as non-features [symbol] .

However, unless our collection V of texts is truly monumental, it is likely to happen that, at least for some triples of typical gardening words such as ‘rose’, ‘lawn,’ or ‘watering’, there is no text in V that contains exactly these three words. The reason is that the set of typical gardening words is too diverse: there is no ‘core’ of them such that, for any three of these, we would find in any reasonably large collection of gardening texts one that contains them all.²⁶ ²⁶ This was different in our chair example in Chapter 2, where any sizable collection of chairs would have at least one chair, indeed many, that has four legs, a flat surface to sit on, and a nearly vertical back.

What does this mean for our tangles? It means that a set S of potential features that each correspond to the frequency of a particular word may have no consistent specification at all, and hence no tangle—even if half our texts in V are about gardening, the other half about baking, and all the features in \vec{S} are either gardening or baking terms.²⁷ ²⁷ One might think that making the words chosen for S less specific should help. But changing ‘watering’ to ‘water’ will not help us find a baking tangle separate from the gardening tangle. And besides, in the intended application we do not know (yet) that gardening and baking will define types of our texts: we are trying to use tangles to find these types!

In Chapter 6 we shall discuss other ways of defining S in such cases, which will help us in Chapter 8.2 to construct text tangles to identify topics, genres—even authors—after all.

3.3 DNA and Protein Tangles: Recognizing Organisms from Imperfect Data

In this example, V is a set of DNA molecules, and our aim is to determine which species, subspecies, or individual organisms they represent.²⁸ ²⁸ One can investigate protein sequences in the same way. We consider the DNA sequences here for simplicity, just to indicate the basic ideas.

In each of our molecules we consider both of its two strands of nucleotides, each in the usual 5′ to 3′ direction. The nucleotides in each strand then have well-defined positions, indicated by integers, and at every position we have one nucleotide. Each of these has one of four possible bases in it, adenine, thymine, guanine or cytosine, which we denote by their first letters A, T, G and C. Every strand thus gives rise to a sequence of these bases, one at every position. The two sequences coming from the opposite strands of the same molecule can be obtained from each other by replacing each base with its partner base and reversing the direction: we call such a pair of sequences inverse to each other.

Very roughly, when we have two identical base sequences obtained from one sample, they will most likely come from the same organism. If we have two similar sequences, they may come from different organisms of the same species. More generally, the more similar two sequences are the more closely related are the species of the organisms from which they were taken.

Since for every base sequence we also have its inverse sequence in our sample—remember that we took both strands from each molecule—the converse implications to those above will hold only up to inverting sequences: two sequences from the same organism should be identical up to inverting them,²⁹ two sequences from organisms of the same species should be similar either to each other or to each other's inverse, and so on. ²⁹ In other words: cither they are equal or each is equal to the inverse of the other.

Not all the positions will be relevant for distinguishing the species which our molecules represent, and we consider only those positions that are. Let us denote by V the set of base sequences, trimmed to these relevant positions, which we obtained from our DNA molecule.

The characteristic features of the elements V are which base is present at each position: we can reconstruct the DNA molecule that gave rise to v from this information. These features thus have the form \vec{s}=\vec{s}(p, B) to tell us that in position p we find the base Bϵ{A, T, G, C}. By our convention, the feature \vec{s}= [symbol] (p, B) then encodes the information that the base at position p is not B but one of the other three bases.

What then, is a tangle of the corresponding set S of potential features? Formally, it will be a consistent specification of the elements of S, a choice of either \vec{s} or [symbol] for every s such that each combination of up to three features it specifies can be found in at least one vϵV, which moreover avoids certain sets [symbol] of features that we may choose. What, then, shall we choose as [symbol] ?

For a start, we will probably wish to include in [symbol] , for some integer n, the set [symbol] ₙ defined at the start of this chapter: this will ensure that each triple of features in a tangle can be found not just in one of our molecules but in at least n of them, which makes the tangle more typical for V the large we choose n.

In addition we can use [symbol] to ensure that the collection of features encoded in a tangle matches the kind of DNA we wish to investigate. For a start, we can prevent a tangle from assigning more than one base to a given position by including in [symbol] all the sets {\vec{s} (p, B), \vec{s} (p, B)} where p is some fixed position but B and B′ denote different bases. Similarly, we can force a tangle to choose at least one base for each position p by including in [symbol] the sets { [symbol] (p, A), [symbol] (p, T), [symbol] (p, G), [symbol] (p, C)} for all positions p. If we wish to focus our investigation on organisms that have some known bases in certain positions, we can further include in [symbol] some sets of features that specify different bases at such positions, thereby ruling out DNA from organisms we wish to ignore.

With such a set [symbol] in place, we may informally think of a tangle τ of S as assigning to each position a unique base, so that this assignment is typical for the molecules encoded in V to a degree determined by the parameter n we chose. If τ has a witnessing set X⊆V, then τ will be typical for the sequences in X in the stronger sense that in every position at least 80% of the sequences in X have at that position the base specified by τ.

For every organism of which our sample contains sufficiently many molecules we can expect to have two tangles: one resembling each strand of a DNA molecule from this organism. Each of these tangles will be a concrete hypothetical base sequence that is typical for the sequences in V (as above), and these two tangles will be sequences that are inverse to each other.⁺ In the language of Chapter 2, these two tangles will be ‘ideal’ copies of (the relevant sections of) the two strands of a DNA molecule from this organism: every actual strand from this organism in our sample may deviate slightly from this, due to some corruption of our data, but the ‘ideal’ base sequence will still be identified.³¹ ³⁰ Remember that the inverse of a sequence is taken not just by reversing it but also by replacing every base with its partner base.³¹ Hence, just like the two strands of a given DNA molecule. these two tangles describe the same thing: we might just pick one of them, knowing that the other can be recovered from our pick by inversion. However, since we have no way of specifying a default strand in a DNA molecule without first identifying the species which it represents (and then checking it against the usual databases), which is part of what we are hoping to use tangles for, we had to keep both strands. This now shows in that our tangles will likely come in pairs, but we may discard one of each pair if desired.

If tangles of S correspond to individual organisms in this way, how are species or subspecies represented as tangles in this setup? The answer is simple: by tangles of subsets of S. For example, a given species would be identified by those DNA positions at which we expect the same specifications across all organisms of that species, but where other species might have different specifications. As with individual organisms and tangles of the entire S, a tangle of this subset of S will be an ‘ideal’ (partial) sequence of bases as they typically occur in DNA of that species. This tangle can be recognized from our sample’ of DNA even if no molecule in that sample has exactly these specifications in those positions (e.g., because our data is imperfect), since tangles are robust against changes of specifications v(s) for just a few vϵV, or even against changes of some v(s) for many v as long as not too many of them happen to the same s.

As an application of the first main tangle theorem, we shall see in Chapter 8.3 how the tangles at different levels interact. Tangles representing various subspecies of a given species, for example, will include the tangle representing that main species as a subset (of features). The theorem will show how all these tangles are organized in a tree-like structure. From this structure we can read off the phylogenetic tree of all the species, subspecies, organisms and so on that are represented in our DNA sample.

The second main tangle theorem will provide verifiable evidence proving, for example, that no single organism has more than a specifiable number of molecules in the sample—information that might be relevant in forensic contexts.

3.4 Drug Development: Chance Discovery or Focused Effort?

Innovation in pharmaceutical research sometimes benefits from chance discoveries: newly found or developed substances are observed to have properties that were, perhaps, not intended in the context in which these substance were studied, but which could be harnessed to beneficial effect in other contexts. In this section we look at how tangles can help with target-driven research by singling out groups of targets that can be represented by a tangle and, in that way, be addressed together.

Suppose we are tasked with developing drugs against some types of harmful pathogens, so many that we cannot target them individually. We might then look into ways of combining these pathogens into clusters: groups of similar pathogens that might be treated by the same drug, so that we only have to develop one drug for each group.

In order to know which pathogens we should put in the same cluster, we thus need a sensible notion for pathogens of being ‘similar’ Now clearly, it may seem, two pathogens are similar if they share many features—features of whatever kind may be relevant for the development of suitable drugs.³² ³² Think of molecular composition, shape or whatever; they do not have to be of any common type.

A moment's thought, however, shows that there is a snag here: we might well end up with clustering together pathogens that are, indeed pairwise similar in that every two of them share many relevant features, but which features these are might depend on the pair of pathogens we are looking at. We might then be able to develop a common drug for every two of those pathogens, but there would be no guarantee that any of these would work for more than two pathogens in the same cluster, let alone all of them.

We are thus thrown back to the beginning: in order to be able to, potentially, treat our many types of pathogens by just a few drugs, we need to cluster not the pathogens themselves but their features. More precisely, we would like to find a few groups of features that can each be targeted by one drug, and such that most of our pathogens have their own features represented sufficiently well by the features in one of these groups that the drug developed for that group works for this pathogen.

Applying the same standard clustering approach to features rather than pathogens, we would now be looking at what makes two features similar—so that they can be clustered together. These aspects of similarity, such as molecular composition or shape, may as such be independent of our pathogen sample. But in order to help with our task, our notion of similarity must also be borne out by our existing sample of pathogens in that features are regarded as similar only if they often occur together. If we ignore this, we shall end up with too many clusters and will need to many drugs.

Tangles offer a possible solution: they are groups of features that often occur together in the pathogens at hand. Although there is no guarantee that this happens always, we can also expect that, conversely, most of our pathogens are represented reasonably well by a tangle that any drug developed for the features in that tangle also works for them.³³ ³³ See Chapter 7.4 for more on how to find among the tangles of S one that best represents the features v(S) of a given element vϵV.

4. Examples from the Social Sciences

This chapter is written using the informal notation from Chapter 2. We have a set V of ‘objects’, and a set \vec{S} of ‘features’ of these object. These features come in pairs s={\vec{s}, [symbol] }, which we call ‘potential features’. The features \vec{s} and [symbol] are the two specifications of s. Every vϵV has one of the features \vec{s} or [symbol] for every sϵS, but never both; we denote the one it has by v(s) and call it v's specification of s.

When V is a set of people, S can be thought of as a questionnaire, with \vec{s} and [symbol] as the two possible answers for a given sϵS.³⁴ ³⁴ Questions with more than two possible answers can be modelled as several questions of this simple yes/no type. We shall discuss this later in more detail.

A specification of S is a choice of either \vec{s} or [symbol] for each sϵS It is consistent if for every three of its choices, \vec{r}, \vec{s}, \vec{t}, say, there exists an object vϵV that specifies r, s and t in exactly the way v(r)=\vec{r} and v(s)=\vec{s} and v(t)=\vec{t}. A consistent specification of S is typical for V if it has no subset in some collection [symbol] of subsets of \vec{S} which we shall specify in every context.

A tangle of S is a typical specification of S, one that is consistent and has no subset in [symbol] .

4.1 Sociology: Discovering Mindsets, Social Groups, and Character Traits

Mindsets were the example of tangles we discussed in the introduction, where we have a set V of people polled with a questionnaire S.³⁵ We may assume for our model that S consists of yes/no questions: if it does not, we can still use it, and simply translate its answers into answers of an equivalent imaginary questionnaire of yes/no questions, which we then use as the basis for computer our tangles.³⁶ ³⁵ In the language developed since then, the mindsets discussed there were tangles with witnessing sets. From now on, we shall use the word ‘mindset’ for any tangle in a questionnaire scenario, regardless of whether that tangle has a witnessing set.

Specifying the collection [symbol] of ‘forbidden’ sets of features is our main tool for determining how broad or refined the mindsets will be that show up as tangles. If we forbid no feature sets, i.e., leave [symbol] empty, then every specification of S returned by one of the people polled will determine a tangle—because it will be consistent and hence typical if [symbol] =0.³⁷ ³⁷ This is not the same as saying that all possible specifications of S will be consistent (and hence typical). Specifications that contain an inconsistent triple will be inconsistent even then, and hence not be tangle. But these specifications are not among those returned in the poll recall how consistency and inconsistent triples were defined.

Such tangles would be too ‘fine’ to be helpful. If we do not wish to influence which tangles are returned by our algorithms other than by determining how ‘broad’ the mindsets they define should be, we can define [symbol] as in Chapter 2.3: as the set of fill triples {\vec{r}, \vec{s}, \vec{t}} shared by no more than n people polled, where n is a number we can experiment with at the computer and see how it influences the numberer (and fineness) of tangles it returns.

But we can also decide to add some sets of features to [symbol] that we think of as inconsistent because of what these features mean. What these are will be up to us: we might add sets of features that are intuitively inconsistent, or sets of features that will occur together as answers on the same questionnaire only if someone tampered with it or tried to influence the survey.

We might even use [symbol] to deliberately exclude some types of mindsets from showing up as tangles, e.g., mindsets we are simply not interested in. In order to exclude such mindsets we simply add to [symbol] the feature sets that make them uninteresting: then no specifications of S that include these feature sets will show up as tangles.³⁸ Of course, subsets we forbid for this reason must be specific enough that they occur only in mindsets that are not of interest to us. ³⁸ For example, we might be interested in the opinions about hooligans among football crowds, but want to exclude hooligans themselves from this survey. Since we may not be able to identify them when we hand out, the questionnaires, but know some answer patterns they are likely to give, we can add these patterns to [symbol] to ensure that, the tangles found are mindsets of spectators that are not themselves hooligans.

We already discussed in the introduction what tangles mean in this example—namely, mindsets—and what the two main tangle theorems offer: a small set of critical questions that suffices to distinguish all the existing mindsets and on which predictions can be based, and verifiable evidence that no mindsets other than those found exist, possibly none. In Chapter 9.1 we shall add another aspect of the tree-of-tangles theorem: it enables us also to structure the mindsets found hierarchically, into broader mindsets and more focused ones refining these.

Discovering social groupings (not groups, see below) is similar to discovering mindsets, except that S is different now: it can still be thought of as a questionnaire, but the ‘questions’ it contains may be answered by an observer rather than the subjects vϵV themselves. Thus, its specifications \vec{s} will be more like the ‘features’ discussed in our furniture example in Chapter 2. What makes tangles special in this application is again that they can identify such groupings without identifying actual groups in terms of their members: tangles are patterns of behavior (etc.) often found together, not groups of similar people.

While this may sound like a truism, it does differ fundamentally from the classical approach that seeks to find groups as clusters of people. Being defined directly in terms of the phenomena that define social groups, rather than indirectly in terms of the people that display them, tangles bypass many of the usual problems that come with traditional distance-based clustering, such as the fact that an individual is likely to belong to more than one social group. However, tangles can help even in finding those groups—which takes us on to character traits.

Imagine we wish to use tangles for a matchmaking algorithm. As before, they can help us to identify, from the answers people have given to a questionnaire S of character-related questions, some combinations of traits that are typically found together.³⁹ Thus, tangles in this context will be ‘types of character’. ³⁹ We have done this with data designed to test for the ‘big five’ personality traits, and got some rather surprising results; see [4].

However, for our matchmaking algorithm it is not enough to know such types: it will also have to match individuals. So we will need a metric that tells us which pairs of people are ‘close in character’—assuming that matching like individuals is our aim (which, of course, can be disputed). The simplest example of a distance function on the set of people that returned our questionnaire would be to consider two people as close if they answered many questions identically.⁴⁰ But tangles can be used to define more subtle distance functions. We shall discuss some of these in Chapter 7.3 and refer to them when we revisit the topic of matchmaking in Chapter 9.1. ⁴⁰ For mathematicians: this is the Hamming distance in the hypercube 2^{S}.

To summarize, let us emphasize again what makes the tangles approach to the study (and discovery) of mindsets, social groupings, or traits of character different from traditional approaches.

It is that we can mechanically find these mindsets (etc.) from observation data without any prior intuitive hypothesis of what they may be, and that we can find them without having to group the people observed accordingly. However, knowing the tangles can then help us also to determine these groups; this will be discussed further in Chapter 7.4.

4.2 Psychology: Understanding the Unfamiliar

In a way, this example is no more than a special case of the mindset example from Section 4.1. But the special context may lend it additional relevance.

While it is interesting to search for combinations of, say, political views that constitute hitherto unknown mindsets that can have an impact on political developments, it is not only interesting but crucial to try to understand minds that work in ways very different from our own. This is particularly relevant in doctor-patient relationships where the doctor seeks to offer an individual with such a different mind a bridge to society, or even just to their particular environment: to enable them to understand the people around them, and to help these people to understand them.

Tangles can already help bridge this gap at the most fundamental level, the level of notions into which we organize our perceptions. We all have a notion, for example, of ‘threat’. But some patients' notion of threat may be different from ours: they may be scared by things we would not see as threatening. And what these are may well come in types: typical combinations of perceptions of everyday phenomena which, for people with a certain psychiatric condition, may combine to a perception of a threat, and thereby form their notion of ‘threat’ that may well differ from ours.

Tangles arc designed to identify such types. The set S would consist of various possible perceptions that experience has shown are relevant, and a tangle would identify the combinations of these that are typical in the sense of Chapter 2.3. The definition of ‘typical’ can be made sufficiently flexible to allow tangles to capture notions, unfamiliar to us, that consist of perceptions that typically occur together in some patients. Once these have been made explicit—recall that every tangle of S will be one specific set of possible perceptions—we can train our intuition on them in an effort to understand our patients rather than just collect lists of unrelated symptoms. We shall get back to how tangles can help identify meaning in Section 4.3.

At a higher level, tangles can help to identify psychological syndromes as such, and maybe discover hitherto unknown syndromes. Indeed, psychological syndromes appear to be exactly the kind of thing that tangles model: collections of features that often occur together.

This would be true for any medical condition, or even for mechanical ‘conditions’ that lead to the failure of a machine. And indeed, there is a corresponding application of tangles for such cases, where they are used to build expert systems for medical (or mechanical) diagnosis [1]. But what makes psychiatric conditions even more amenable to the use of tangles is that the symptoms of which they are combinations are so much harder to quantify. Tangles come into their own particularly with input data that cannot be expected to lie reliably precise.

So how would we formalize the search for hitherto unknown psychological syndromes? Our ground set V would be a large pool of patients in some database. The set \vec{S} would be a set of possible symptoms. Tangles will be collections of symptoms that typically occur together: medical, or psychological, conditions or illnesses.

We shall see in Chapter 9.2 that the set T of tangle-distinguishing features returned by the tree-of-tangles theorem will consist of ‘critical’ symptoms or combinations of symptoms that can be tested on a patient in the process of diagnosis. Every consistent specification of T defines a unique tangle: a unique condition that has all the symptoms in this specification of T. In most cases, this will be the correct diagnosis.

Note, however, that our emphasis here is on finding psychiatric conditions: our aim is to discover combinations of symptoms that constitute an illness. It is not on diagnosing a given patient with one of these conditions (although checking the symptoms in T can play an important part on this), but on establishing what are the potential conditions to look for in the diagnosis. It seems to me that a lot has happened in psychology here in recent years, in that conditions are now recognized as illnesses that were not even thought of as typical combinations of symptoms (i.e., tangles) not so long ago.

4.3 Analytic Philosophy: How to Quantify Family Resemblances

The aim of this section is to indicate how tangles can help us identify meaning. Inasmuch as meaning is constituted in a social process this discussion belongs within the scope of this chapter. It also has direct implications for the teaching of languages: in education, but also in machine learning which might place it in Chapter 5. In Chapter 10.3 we shall discuss an application in which tangles identifying the meaning of a word are used to steer a user of an interactive thesaurus towards the desired word.

To keep the discussion focused we shall concentrate on the meaning of words. There are obvious wider analogues, of course, and indeed the relevance of social aspects to the constitution of meaning will be more relevant still when we talk about the meaning of entire phrases, perhaps depending on their contexts, or the meaning of behavior in non-verbal communication.

Meaning of words, however, is not only easier to talk about: we have already done much of this when we discussed the furniture example in Chapter 2. There we observed that tangles can identify types of furniture such as chairs, tables and beds, from lists of features of concrete specimen.

This contrasts with the naïve approach of trying to define words by a list of predicates, as classical dictionaries used to do. The idea there is that something warrants being referred to by that word if and only if it satisfies all the predicates on the list.⁴¹ In other words, the list should be long enough that no things other than those we want to use our word for have all these properties, and it should at the same time be short enough that, conversely, all the things we wish to name by our word do indeed have all the properties from the list. ⁴¹ . . . or, more generally, satisfies some logical formula in terms of these predicates

Put another way, if we assume that every predicate mt the list describes a well-defined set of objects⁴² we would like the set of objects to be referred to by the word we are trying to define to be exactly the intersection of those sets: no larger and no smaller. As the furniture example demonstrates well, such a list is unlike to exist: it is probably impossible to come up with a list of potential features (or furniture) such that the things that have all these features are precisely all of the chairs. ⁴² This is another dilemma with this naïve approach: it requires that there exists a hierarchy of predicates, where some are defined ‘before’ others and can hence be used in the list for their definition. Tangles require no such hierarchy.

The same problem arises more generally if we try to define the meaning of a word by any logical formula of previously defined predicates.

Wittgenstein [5] recognized this dilemma and argued that meaning cannot be captured by a simple taxonomic approach. Instead, he argued, the things referred to by a word behave more like members of a family: individuals who resemble each other but cannot be identified by any list of features that precisely they share.

Tangles offer a way to quantify Wittgenstein's family resemblances.⁴³ The chair example in Chapter 2 describes how. ⁴³ Thanks to Nathan Bowler for pointing this out to me.

In particular, tangles show that the intrinsic extensional imprecision in most attempts to define the meaning of a word in terms of known predicates simply by way of a logical formula does not make it futile to look for precise alternative ways to define meaning (in other ways than by logical formulas).⁴⁴ As we shall see, there are a number of parameters we can use to quantify the notion of a tangle.⁴⁵ So even if we take the view that the notion of ‘meaning’ can be formalized as ‘tangle of predicates’, as I would argue, there will not be one ultimate such notion. ⁴⁴ It seems that this difference has occasionally been overlooked.⁴⁵ For example, by choosing the parameter n from our discussion of the term ‘typical’ in Chapter 2.3—we shall meet this again in the sets [symbol] . in Chapter 6.2—or by choosing an order function as described in Chapter 6.5.

Rather, there are many precise ones, one for every choice of our quantitative tangle parameters. Then in every ‘context’ S, i.e., for every list \vec{S} of predicates deemed relevant to the definitions we are trying to make, every tangle of S constitutes an instance of that notion of ‘meaning’: a potential definition of what one particular word means.⁴⁶ Each of these notions will be completely precise: tangles are a precise way to define extensionally imprecise meanings of words. ⁴⁶ In our earlier furniture example, these were the meanings of ‘chair’, ‘table’, ‘bed’ and so on. By our choice of quantitative tangle parameters we can influence how broad or narrow that meaning is going to be.

After this rather abstract discussion let us return once more to our concrete example, and summarize how exactly the notion of ‘chair’ is given by a tangle. We start with a list S of potential features of furniture. Any tangle of S is one particular specification of S: a choice of either \vec{s} or [symbol] for every sϵS, either confirming the feature \vec{s} or confirming its converse [symbol] . The combination of features that we might think of as describing ‘the perfect chair’ would be a tangle—no matter whether such a chair exists in the world or not.

Although every specific tangle in this context is a concrete list of predicates, the notion of ‘tangle’ as such is purely formal and makes no reference to such predicates: a tangle is a specification of S that satisfies certain formal requirements consistency and being typical—which say that it must not contain certain small sets of features (which in our context are predicates). These requirements ensure that tangles define ‘good’ notions rather than ‘bad’ ones. The settings of certain parameters in these requirements also determine whether the corresponding tangles capture board or narrow notions.

Thus, whether or not a combination of potential features (a specification of S) is a tangle is decided by a precise definition. This does not imply, however, that it must be clear for every piece of furniture whether or not it ‘belongs to’ a given tangle, e.g., whether it is a chair. Indeed this is not clear for real pieces of furniture in the real world, and it is one of the strengths of our approach that tangles offer a precise way to capture even extensionally imprecise notions such as this one.

4.4 Politics and Society: Appointing Representative Bodies

As discussed in Chapter 2, the tangle approach to clustering is that we do not primarily seek to divide our set V into groups based on similarity between its elements. This is true even if similarity is measured by how the elements of V specify S, in which case we might group u, vϵV together if they specify many sϵS in the same way (as \vec{s} or [symbol] ): if u(s)=v(s) for many sϵS.

Rather, determining tangles is more about grouping features: not in the simple sense that we find traditional distance-based clusters in the set \vec{S}, but in that we look at how the elements of V specify S and find particular such specifications that are typical for the elements of V. In the mindsets application from Section 4.1, for example, tangles are ways of answering all the questions in S that are typical for how the various vϵV answered them.

Once we have found the tangles of S, however, we can use them to group the elements of V after all. In terms of the mindsets scenario, on which we shall base our further discussion here for better intuition, we would thus be looking for ways to group the elements of V according to their mindsets.

This can be done in various ways, which we shall look at in Chapter 7.4. For example, we might associate each vϵV with the mindset τ of S that represents its views best: the tangle τ of S for which the number of elements s of S that τ specifies as v does is maximum. Conversely we may seek, for each mindset τ found, the person vϵV closest to τ in this sense, and think of these v as best representing the views held amongst the members of V.

If we add our assumption from Section 4.1 that V was chosen so as to represent some larger population P well, we shall then have found a small group of people that ideally represent the views held in our population P on matters explored by S. This process might be used, then, to appoint delegates to a body whose brief is to make decisions likely to find maximum consensus in P.

This contrasts with the usual democratic process of electing the delegates by majority vote. In a first-past-the-post system with constituencies, this can generate parliaments with large majorities even when, in each constituency, the majority of the successful candidate was small but the population is homogeneous enough across constituencies that in most of these the successful candidate is of the same political line.

In systems with proportional representation this is avoided, but such systems require the previous establishment of political parties. Even when these exist, they may have developed historically in contexts that are less relevant today. Finding the tangles of a political questionnaire is like finding the political parties that ought to exist today for the elections at hand People vϵV representing these virtual ‘parties’ as described above could then be delegated to our decision-making body straight away.

If we wish to appoint more delegates for tangles, or virtual parties, with a larger following, we could elect representatives from these virtual parties by a standard vote using proportional representation. Alternatively we could simply refine this tangle into smaller tangles (see Chapters 6.3 and 9.3), so that all tangles end up with roughly the same amount of support and could thus be represented by one delegate per tangle.

This approach may be even more relevant outside politics, where there are many situations on a smaller scale in which we seek to appoint a decision-making body. This might be the governors for a school, or a steering committee for a choir. At such a smaller scale there will be no constituencies, and there may be no established parties relevant to the brief of that body. But appointing for every tangle τ of S the member of V (amongst those willing to stand) whose views on S are closest to that tangle would produce a committee likely to represent the views held in V well.

4.5 Education: Combining Teaching Techniques into Methods

This application starts from the assumption that different teaching techniques work well or less well with different students: that a given technique is not necessarily better or worse than an alternative for all students at once, but that each may work better for different sets of students.

Ideally, then, each student should be taught by precisely the set of techniques that happen to work best for him or her. Of course, this is impractical: there are so many possible combinations of techniques that most students would end up sitting in a class of their own. But our aim could be to group techniques into, say, four or five groups, to be used in four or five classes held in parallel, so that each student can then attend the one of these four or five classes whose techniques suit him or her best.

The question then is: how do we divide the various techniques into the groups that correspond to the classes? Just to illustrate the problem. consider a verve simple example. Suppose first that some students benefit most from supervised self-study while others are best served by a lecture followed by discussion in class. Suppose further that some students understand a grammatical rule best by first seeing motivating examples that prepare their intuition, while others prefer to see the rule stated clearly to begin with and examples only afterwards. So there are four possible combinations of techniques, but maybe we can only have two classes. So we have to group our techniques into pairs.

How shall we select the pairs? Shall we have one class whose teacher lectures and motivates rules by examples first, or shall we group the lecture-plus-discussion class with the technique of introducing rules before examples?

This is where tangles come into their own. Think of V as a large set of students evaluated for our study, and of \vec{S} as a set of teaching techniques. Every tangle of S will be a particular combination of techniques (a specification of S) which, in the language of our furniture example in Chapter 2 (where \vec{S} consists of features) ‘typically occur together’. What does this mean in our context?

What it means formally depends on the definition of ‘typical’ that is determined by our choice of the set [symbol] introduced in Section 4.1. Informally it implies, for example, that if the tangle is witnessed by a set X∥V (see Chapter 2.4) then this X is a group of students that would benefit particularly well from the combination of techniques specified by the tangle: for every technique in that tangle, a majority of the students in X prefer that technique to its converse. Thus, X would be an ideal population for the class in which the techniques from this tangle are used, while a set Y witnessing another tangle would consist of students best served by the teaching techniques specified by that tangle.

Once more, speaking about tangles in terms of witnessing sets helps to visualize their benefits, but it is not crucial as such: the benefit arises from finding the tangles as such, and setting up those four or five classes accordingly. We then know that this serves our students best collectively—and can happily leave the choice of class to them.

Let us, from now on, refer to tangles of teaching techniques as teaching methods: combinations of techniques that work particularly well together for some students.

When we return to this example in Chapter 9.3, we shall have to address the question of how to set our parameters in such a way that, even if the set \vec{S} of potential teaching techniques is large, we still end up with a desired number of tangles four or five in our case—that may be dictated by the formal environment, school etc., in which the teaching is taking place. We shall also see how, if we do not wish to let the students choose their class themselves, the tree-of-tangles theorem can help us devise an entry test that assigns students to the class that benefits them best.

4.6 Economics: Identifying Customer and Product Types

The context of economics offers a wide range of interesting choices for our set V of ‘objects’ studied together with some potential ‘features’ listed in a set S. This is illustrated particularly well by a pair of examples described below, which describes two complementary, or ‘dual’, aspects of the same scenario.

In our first example, let V be a set of customers of an online shop, and S the set of items sold at this shop. Let us assume that each customer v makes a single visit to the shop, specifying sϵS as \vec{s} if v includes s in his or her purchase, and as [symbol] if not. We shall think of the specification v(S) of S as v's ‘shopping basket’, dividing as it does the set of items into those bought and those not bought.

An arbitrary specification of S, then, is a hypothetical shopping basket, and a tangle of S is a typical shopping basket for this set of customers.⁴⁷ If we mentally identify a customer v with his or her shopping basket v(S), we may also think of a tangle S as a (hypothetical) type of customer whose ‘features’ are his or her purchases v(s)=\vec{s} and non-purchases v(s)= [symbol] .⁴⁸ ⁴⁷ This is not the same as just a group of items ‘often bought together’, as are already suggested by some online shops today. A shopping basket defined by a tangle is typical in a more subtle way; for example, it is also typical in what it does not include.⁴⁸ This is analogous to thinking of the chair tangle as a type of furniture, or of a mindset tangle as a type of person holding such views. In each case, the tangle is a set of features typical for the elements of V, and we may think of it as a hypothetical ‘typical’ element of V that has exactly these features.

Alternatively, we may think of S, the set of items of our shop, as our set of objects (which would normally be denoted by V, but this letter is taken now), and of the customers in Vas potential ‘features’ of these items: a customer v becomes a feature \vec{v} of precisely those items s that v bought, and a feature [symbol] of those items s that v did not buy. Each item s then defines a specification s(V) of V, specifying those v that bought it as s(v):=\vec{v}, say, and those that did not buy it as s(v):= [symbol] . We may think of s(V) as something like the ‘popularity footprint’ of the item s with the customers in V.

Note that the information encoded here is no different from that described by our earlier setup, where every customer specified each item s as \vec{s} or [symbol] . We thus have two ways now of describing the same set of customer preferences.⁴⁹ ⁴⁹ For mathematicians: there is a formal duality here in that v(s)=s(v) with the obvious interpretations of v as a function S→{0,1} and s as a function V→{0, 1}. The customer preferences described in two ways can he formalized as the edge set of the bipartite graph with vertex classes V and S and edges vs whenever v bought s. This edge set can be described alternatively as a list of neighborhoods of the vertices in V or of those in S.

What are the tangles in our second setup? They arc tangles, or typical specifications, of V: hypothetical popularity footprints with the customers in V that are typical for the items in S. If we mentally identify an item s with its popularity footprint s(V), we may think of a tangle of V as a type of item whose ‘features’ are its fans s(v)=\vec{v} and non-fans s(v)= [symbol] .

Let us look at some examples of both kinds of tangle, and of how they might be used.

Tangles of S are typical (if hypothetical) shopping baskets, collections of items that are typically bought or avoided together. For example, there might be a tangle, or ‘typical shopping basket’, full of ecological items but containing no environmentally harmful ones. Another might contain mostly inexpensive items and avoid luxury ones.⁵⁰ ⁵⁰ In our current informal setup, these would be tangles of subsets of S, not of S itself—for example, of the subset of the ecologically critical or the unusually priced items. When we revisit this example in Chapter 9.5, we shall find a way of capturing all groups of items, such as the ecological or the inexpensive ones, by tangles of the same set.

Note that the ‘opposites’ of these tangles will not normally be tangles. Indeed, a shopping basket full of luxury goods and avoiding cheap items is unlikely to be typical, because customers that like, and can afford, luxury good will not necessarily shun inexpensive items. Similarly, inverting the ecological tangle will not produce another tangle, since there is no unifying motivation amongst shoppers to buy environmentally damaged goods, and probably no unifying motivation to avoid ‘green’ products either.

If we personify these tangles as indicated earlier, and think of the as (hypothetical) people putting together these hypothetical shopping baskets, we could think of our second tangle as the budget-oriented customer type, one that prefers inexpensive brands and cannot afford or dislikes unnecessarily expensive ones, and of the first as the ecological type that prefers organic foods and degradable detergents but avoids items wrapped in plastic. And, crucially, our tangle analysis might throw up some unexpected customer types as well—perhaps one that prefers items showing the picture of a person on the packaging.

Applications of tangles of S might include strategies for grouping goods in a physical shop, or running advertising campaigns targeted at different, types of customer.

Tangles of V, on the other hand, are typical popularity footprints, ways of dividing V into fans and non-fans that occur for many items simultaneously. If what interests us about the items in S is mainly how they appeal to customers, we may think of tangles of Vas types of items.

For example, assume that the ecological goods in our shop are substantially more expensive than non-green competing goods. Then V splits neatly into a ‘green fan set’ of customers, those likely to buy these green items even though they are pricier, and the rest of all customers. who will avoid them because they are more expensive. This division of V, or hypothetical popularity footprint, is borne out in sufficient numbers⁵¹ by the green items in S to form a tangle of V, since these items are both liked by the ‘green’ customers and disliked by the others for their price. ⁵¹ This is a reference to the number n used in Chapter 2.3 to define tangles informally. It will be referred to again when we define tangles formally in Chapter 6.2.

An application of finding tangles of V could be to set up a discussion forum for each tangle and invite the fans for that tangle to join. Since they have shown similar shopping tastes, chances are they might benefit more from hearing each others views than could be expected for an arbitrary discussion group of customers.

One last remark, something the reader may be puzzling over at this point. Since our two types of tangle are ‘dual’ to each other, it so happens that witnessing sets for the first type are the same kinds of sets as tangles of the second type: they can both be thought of as sets of customers. But they are not the same. A set X⊆V witnessing a tangle of S is a set of customers such that for every item sϵS either most of the people in X bought s (if the tangle specifies s as \vec{s}) or most of the people in X did not buys (if the tangle specifies s as [symbol] ). By contrast, a tangle of V (thought of as the set of those v which it specifies as v)⁵² is a group of customers such that every three of them jointly bought some sizable set of items (at least n).⁵³ ⁵² Unlike in other contexts, every v in this example has a default specification \vec{v} the set of purchases of v rather than his non-purchases. The set {\vec{v}|vϵV}, therefore, is well defined.⁵³ Indeed, more is true: for every three customers v, regardless of how the tangle τ specified them, there exists a set of at least n items that are each bought by v if r(v)=\vec{v} and not bought by v if r(v)= [symbol] .

Put more succinctly: in the first case, most people in some set of customers (not too few) agree about every single item, while in the second case, the tastes of every few (e.g., three) customers are witnessed by some common set of items (not too few), regardless of whether their tastes coincide on these items or not.

5. Examples from Data Science

This chapter is written using the informal notation from Chapter 1.3, rather than that of Chapter 2. We have a set V of ‘points’, and a set S of partitions {A, B} of V into two sides, the disjoint subsets A and B.

We usually denote the sides of a partitions s as \vec{s} and [symbol] , so that s={A, B}={\vec{s}, [symbol] },⁵⁴ think of the arrows as ‘orienting’ s towards the sides they denote, and write \vec{S} for the set of all sides of elements of S. Given any sϵS, every vϵV lies in exactly one of the sets \vec{s}. [symbol] (because they partition V); we denote this side of s as v(s). ⁵⁴ Note that this notation does not fix which of the sides A, B is \vec{s} and which is [symbol] .

An orientation of S is a choice of one side of s for every sϵS: a subset of \vec{S} that contains exactly one of \vec{s}, [symbol] for every sϵS. An orientation τ of S is consistent if every three of its elements have a non-empty intersection. For example, every orientation of S of the form v(S) for some vϵVis consistent. A consistent orientation of S is typical for V if it has no subset in some collection [symbol] of subsets of \vec{S} which we shall specify in every context.

A tangle of S is a typical specification of S, one that is consistent and has no subset in [symbol] .

5.1 Indirect Clustering by Separation

In Chapter 1.3 we considered ‘bottlenecks’ of data sets, and partitions ‘at’ such bottlenecks. Formally, we shall have a lot of freedom to specify exactly which partitions of our set V are deemed to be such ‘bottleneck partitions’. This will depend on the context in which our clustering is taking place; one example is given in Section 5.2 below. But there are also some generic approaches that can be applied in many contexts; see Chapters 6.4 and 10.1.

For now, let us simply think of the bottleneck partitions of V as precisely those that are in S, whatever they may be in a given context, and use the formal definition of consistency for orientations of S given above. In particular, we make no assumption about the sides of bottleneck partitions (those in S): these sides can be large or small, and contain a ‘point cluster’ or not. Indeed it is important that S must be definable without reference to point clusters, because it is our aim to define clusters via tangles, not the other way round.

At the same time, we shall continue to appeal to the intuitive picture from FIG. 3, where the bottleneck partitions include all those that divide the point set at the ‘handle’, an elongated isthmus between one duster on the left and three on the right.⁵⁵ ⁵⁵ Note, however, that it is possible even in this example to define S, the set of ‘bottleneck partitions’, without any reference to point clusters—unlike the way we did it in Chapter 1.3. For example, we might take S to be the partitions {A, B} of V for which there exists a short straight line [symbol] such that, for some small distance δ, we can connect any two points in A by polygonal paths whose vertices lie in A and whose straight line segments between these vertices have length at most δ and do not cross [symbol] , we can connect any two points in B in the same fashion, but we cannot connect any point in A with any point in B by a polygonal path with vertices in V and line segments of length at most δ not crossing [symbol] . With such a definition, clumsy as it is, we could prove that each of the four point clusters lies mostly on one side of every partition in S, and thus defines an orientation of S (indeed a consistent one).

Let us first consider the extreme case that S contains all the partitions of V. In this case, the orientations v(S) of S are the only consistent ones. Indeed, consider any consistent orientation τ of S, and let \vec{s}=A be the smallest element of τ in terms of |A|, the number of points it contains. Note first that A≠0: being consistent, τ cannot contain the set {A, A, A} if the intersection of its elements is empty, which it is if A=0. So A contains a point, v say.

Suppose A contains another point u. Then A′=A\{u} is a side of a partition in S, the partition s′={A′, V\A′}.⁵⁶ As A′ is smaller than A, we cannot have A′=:\vec{s}ϵτ by our choice of A, so [symbol] ′ϵτ. Similarly, {v}=:\vec{r} cannot lie in r by the minimality of A, so [symbol] ϵτ. But now {\vec{s}, [symbol] ′, [symbol] }⊆τ while \vec{s}∩ [symbol] ∩ [symbol] =0, contradicting the consistency of τ. Hence our assumption that A contains a point other than v is false, and we have {v}=A=\vec{s}ϵτ. ⁵⁶ Here we are using our assumption that S contains all the partitions of V.

Let us prove that τ=v(S). Given and partition of V, one of its two sides must be in τ. In fact it must be the side containing v, since the other side has empty intersection with {v}, so they cannot both lie in τ. Hence τ orients every partition in S as v does, completing our proof that τ=v(S).

Depending on our choice of [symbol] , this has the following consequences for tangles of this extreme choice of S. If [symbol] =0, the tangles of S are precisely its consistent orientations, those of the form v(S). As soon as we forbid singletons \vec{s}={v} as elements of tangles, however, e.g. by taking [symbol] = [symbol] ₙ with n>1,⁵⁷ or by directly setting [symbol] :={{{v}}:vϵV}, we have no tangles of S at all: since tangles have to be consistent, they can only be of the form v(S), but we have just ruled those out. ⁵⁷ The sets [symbol] were defined in the preamble to Part II.

For this reason, we shall not normally consider as S the set of all partitions of V, but subsets of partitions that divide V in a particularly natural way.

However, our example can teach us something we left unproved in Chapter 1.3: that any tangle τ of FIG. 3 orients all the partitions at the handle in the same direction. The proof is similar to our consistency proof above, and left to the reader.⁵⁸ ⁵⁸ Consider a set S as defined in footnote 55, except that the dividing line t between the two sides of a partition in S need not be straight but must fit in a box of side length the width of the handle. Clearly, τ cannot orient two partitions τ and s at the handle away from each other: then their two sides chosen by τ would have empty intersection, making τ inconsistent. But neither can they point towards each other. If they did, we could cut up the space between them, which is part of the handle, into singletons by further partitions from S, just as we cut up the entire set Vin our earlier example where all partitions of V were in S. Our choice of S now ensures that the partitions we need for cutting up this space are all in S. If singletons {v}⊆τ are ruled out by our choice of [symbol] , which we assume, we obtain the same contradiction as earlier.

5.3 Linguistics: Teaching Computers Meaning, and an Interactive Thesaurus

Our notions determine how we see the world. They are our way of cutting the continuum of our perceptions and ideas into recognizable chunks which, to some extent, persist in time, space, and across different people. Chunks of ideas or perceptions which we bundle together again and again in different places, and which are similar to the recurring chunks in other people's minds.

Inasmuch as we understand, remember, and communicate aspects of the world around us as structures of notions, the question of what these notions are, i.e., which ideas or perceptions we combine into notions, determines what we can understand, remember, or communicate about the world. Since in everyday life⁵⁹ we do not choose our notions consciously, understanding and quantifying them—as tangles enable us to do—is perhaps significant for our own understanding of the world only when we seek to compare how people from different cultures or backgrounds understand the world differently. ⁵⁹ Unlike, for example, in mathematics: there, decisions about which properties of the (mathematical) objects studied should be bundled into notions to facilitate further study are part of our daily bread. And they are as important as elsewhere in life, since they determine what structures of the objects under study become visible and can therefore be explored.

But as soon as we try to teach a computer what our notions are, perhaps through some interactive process,⁶⁰ we need some quantitative definition of a ‘notion’. As we have already seen, tangles may well offer that. ⁶⁰ For example, the computer might learn by showing us pictures of groups of objects and asking which of them ‘do not belong’ in this group.

More ambitiously, if we seek to enable computers to ‘understand’ the world by themselves, the question of what should be their own fundamental notions, not necessarily copying ours, will be on the agenda even more. This is because there are ‘good’ notions and ‘bad’ ones, judged by how they help us understand the world. Good notions cut the continuum of ideas and perceptions along lines running between phenomena which we would like to distinguish,⁶¹ whereas bad notion; cut across such phenomena and are therefore less helpful for distinguishing them. As we saw in Chapter 6, this aim of finding good notions, possibly unlike ours, is very similar to finding tangles seen as specifications of set partitions. ⁶¹ Despite the wording here, this is not entirely a matter of taste. Very crudely, one might say at good notions facilitate the expression of theories that describe the world better than others for example, in the sense of making more specific or substantial predictions.

We discussed in Chapters 2.3 and 4.3 how the notion of ‘chair’ can be captured as a tangle of potential features of furniture. Note that we can choose to be more demanding regarding the quality of this notion by raising the parameter n in the definition of [symbol] ₙ-tangles, since a furniture type will show up as an [symbol] ₙ-tangle τ only if every three of the features in rare shared by at least n items. Another way to be more demanding of an [symbol] ₙ-tangle, in order to ensure it only identifies high-quality notions, would be to replace ‘three’ with some higher number: to ask that not only all triples of features in tangle are shared by many items but, perhaps, all quintuples.⁶² ⁶² If we are too demanding, of course, we shall simply not get enough tangles to serve as notions, so we have to strike a balance here. But the quantitative parameters of [symbol] ₙ-tangles allow for precisely that not to mention the numerous possibilities of other choices off.

For the sake of our chair example, we considered as features only properties of furniture, such as being made of wood, which may or may not apply to a particular piece at hand. However when we try to train a computer to form or recognize notions, there is no reason to be so restrictive: we can also use other parameters that help us distinguish between different notions, such as context, or the time when it was fashionable.⁶³ ⁶³ Imagine a party game where a person thinks of something and we have to guess what it is by asking yes-no questions of any kind. Every such question is a ‘potential feature’.

In Chapter 6.5 we saw how to use order functions to divide potential features into hierarchies, assigning low order to basic features and higher order to more specific ones. This is nowhere more relevant than when we employ tangles to capture notions in our ideas and perceptions: some notions are clearly more fundamental than others, and some questions (potential features) are more basic than other questions and thus should have lower order.

In addition to, or as a basis for, assigning an order to potential features in terms of similarity functions for set partitions (see Chapter 6.5), there are two ways of defining order explicitly that come to mind in the context of meaning: one is relevance, the other what one might call clarity or definiteness.

As an example for a relevance-based order function we could choose to assign all color questions high order in our search for furniture types if we consider color irrelevant to how furniture splits into types. As an example for a clarity-based order function, suppose we are trying to distinguish hairstyles. We are likely to get more helpful answers in the clarity sense (of delineating hairstyles from each other) if we ask when they were fashionable than whether they are pretty, so the former question should receive lower order on the clarity scale than the latter.

Note that these two considerations for how to define an order function may well conflict: being pretty or not is perhaps the most relevant aspect of a hairstyle, but it may also be the least clear in that people have divided opinions about it. Note also that even if we wish to base our order function on relevance or clarity or both, we do not necessarily have to define the order |s|, or a weight w(s), manually for every question s: both relevance and clarity can be gleaned to some extent from how the people who took our survey answered its questions, and therefore computed mechanically.

What we have described so far is how a computer can learn the meaning of words as we use them, or come up with notions of its own that are formed by observing the world. Both these are aspects of what one would call the formation of passive vocabulary. Once this has been achieved, our computer will also want to know which of its tangle-encoded notions best describe a given object presented to it. This is a problem we discussed in Chapter 7.4: the problem of how to match a given object to a tangle of potential features, the tangle that best captures its actual features. See there for further discussion.

Let us complete this section with a straightforward application of tangles of notions, just as an example of what may be possible: in interactive thesaurus for non-native speakers.

Roget's classical Thesaurus helps writers find the best word for what they are trying to say simply by grouping together some likely candidates. The relevant group of words can be found by looking up a word whose meaning is close to the intended meaning, but maybe does not quite capture it. If a word has multiple meanings, it is linked to several such groups.

The value of this lies in offering the writer a relevant choice, but the thesaurus does not help with making this choice. This is fine if the writer knows all the words on offer, and perhaps just could not think of the right one. For learners of a language, however, this falls short of their need: they, too, will know what they are trying to express, but need help in finding the best word for it. Let us see how tangles can help them.

In the simplest model, we could devise for every word field currently offered together as a group of choices a questionnaire S whose answers for any given context would enable us to choose the correct word from this field. Each of these words, then, is likely to correspond to a tangle of S—a tangle we would be able to compute before our thesaurus is published. We could then also compute the tangle-distinguishing small set T of questions from Theorem 1. These questions from T could then be put to the user trying to identify the right, word for their intended notion, and answering just these will steer them to the word that, best fits their intended notion.

Recall that the questions in T may be combinations of questions from S, so the questions a user really has to answer may be a little larger. But the subset of S needed to form the questions in T is still likely to be smaller than S itself. This is crucial for making a good thesaurus: it will matter to the user whether they need five questions or fifteen to be steered to the fitting word.

Devising a questionnaire S for each word field in the thesaurus, and answering all its questions for each word, may look like a lot of work. But this work has to be carried out only once, when the thesaurus is made: it is offset by a gain on the user side in that the questions asked are chosen specifically for each word field selected by the user, and are chosen particularly well for this word field.

In a more sophisticated model, we could grade the questions from S as more or less relevant for the corresponding word field, and assign them an order correspondingly. We would then obtain a hierarchy of tangles as described in Chapter 6.3. This would correspond to a hierarchy of more or less general or specific words, just as in reality.

In this last respect, our thesaurus might even learn from user interaction. Although a user may not know the meanings of the words on offer, they will be able to grade the questions put to them as more or less relevant to their specific search: tangles, after all, reflect notions, not words, and users come with such notions in their minds. Our tangles will then have to be recomputed from time to time when enough user feedback has been collected, and re-checked editorially against the words on offer. At this point, editors might also comment on user-defined tangles that do not have a corresponding word to match: such tangles will exist, since notions exist that arc not exactly matched by words. Compared against the notions in the minds of speakers of other languages, however, this seems even more likely and worth addressing in ongoing editorial work.

Disclosed are a computer-implemented method, a system and a computer readable storage means as mentioned above.

In particular, the computer-implemented method—which in the following is also denominated as method M1—uses the mathematical theory of tangles of abstract separation systems to identify, and distinguish, clusters of qualities (CloQs) amongst potential qualities of given objects, by way of
testing the elements of a given set V of objects to determine which of the qualities in a given list S of potential qualities each of them has;
identifying subsets of S as CloQs: as groups of qualities from S that often occur together in the objects of Vin the sense that they form a tangle of S;
computing a nested set τ of no more than |V| Boolean expressions of qualities from S that suffice to distinguish the CloQs, in that for any two CloQs there exists a combination tϵT of qualities which is shared by the qualities in one of the CloQs but not by those in the other;
if the qualities in S have no CloQ, computing a nested set T of no more than |V| Boolean expressions of qualities from S which, between them, witness that no CloQ can exist in S.

According to advantageous embodiments of said method M₁, the list S is extended to all bipartitions, or separations, s of V of increasing order, where the order of s is lower if s separates fewer pairs of objects in V that are similar, in the sense of any given similarity function, in terms of the qualities in S and higher if s separates more such pairs, so that
the tangles identified, and distinguished, are tangles of the set Sₖ of separations of V of order less than k (which may or may not include the qualities in S of order <k themselves), giving rise to clusters of separations (CloSs) of increasing order that become more and more specific as k grows;
the first set T from M₁ is now one of separations of V, which are efficient in that T contains, for any two CloSs, a separation of lowest possible order that distinguishes these two CloS s in that one of them contains this separation but the other does not;
the second set T from M₁ now witnesses that a set Sₖ of separations of order less than some fixed k has no CloS.

In the following, the method of such embodiment is also denominated as M₂.

As an advantageous embodiment of the method M₁, each CloQ is witnessed by a set U⊆V of objects such that, for most qualities qϵS, at least some fixed portion of the objects in U share the quality q if q is in the CloQ, and share the logical inverse (or negation) of q if it is not.

According to further advantageous embodiments of method M₁, the CloQs are not necessarily witnessed by such sets U⊆V but are witnessed by all the triples, or n-tuples for some fixed n other than 3, of qualities in S in that at least some fixed number of the objects in V share these three qualities or their negation according as these qualities or their negations are in the CloQ.

As an advantageous embodiment of the method M₂, each CloS in Sₖ is witnessed by a set U⊆V of objects such that, for most sϵSₖ, at least some fixed portion of the objects in U lie on the side of s that is specified by the CloS.

According to further embodiments of the method M₂, the CloSs are not necessarily witnessed by such sets U⊆V but are witnessed by all the triples, or n-tuples for some fixed n other than 3, of separations in Sₖ in that at least some fixed number of the objects in V lie on the side of each of these three or n separations that is specified by the CloS.

The method M₁ may be used to cluster the objects in V according to which of the CloQs their qualities match best.

The method M₂ may be used to cluster the objects vϵV according to which of the CloSs best match the set of sides of the separations in S that contain v.

Each of the methods M₁ or M₂ may be used to establish as predictions for the qualities of an unknown object the qualities of a random known object sampled from test results performed on a small set of objects. With the additional provision that the objects for which predictions are sought were tested on the small set T of questions established in M₁, each of the methods M₁ or M₂ may be used to establish which CloQ best represents the given object and can thus be used to predict its qualities.

### REFERENCES

[1] R. Diestel. Tangles in the empirical sciences: indirect clustering by separation. In preparation.
[2] R. Diestel. Abstract separation systems. Order, 35:157-170, 2018.
[3] C. Elbracht, J. Kneip, and M. Teegen. Tangles are decided by weighted vertex sets. Advances in Comb., (to appear).
[4] J. Erde, E. G″urpinar, and R. Diestel. Character traits and tangles. In preparation.
[5] Ludwig Wittgenstein. Philosophical Investigations. Basil Blackwell, Oxford, 1953.

1. A computer-implemented method to use a mathematical theory of tangles of abstract separation systems to identify, and distinguish, clusters of qualities (CloQs) amongst potential qualities of given objects, by way of:
testing elements of a given set V of objects to determine which of qualities in a given list S of potential qualities each of them has;
identifying subsets of S as CloQs: as groups of qualities from S that often occur together in the objects of V in that the subsets form a tangle of S;
computing a nested set τ of no more than 1 VI Boolean expressions of qualities from S that suffice to distinguish the CloQs, in that for any two CloQs there exists a combination t E τ of qualities which is shared by the qualities in one of the two CloQs but not by those in the other of the two CloQs; and,
if the qualities in Shave no CloQ, computing a nested set τ of no more than |V| Boolean expressions of qualities from S which, between them, witness that no CloQ exists in S.

2. The method according to claim 1, wherein S extends to all bipartitions, or separations, s of V of increasing order, where an order of s is lower if s separates fewer pairs of objects in V that are similar, in the sense of any given similarity function, in terms of the qualities in S and wherein the order of s is higher ifs separates more pairs of objects in V that are similar, so that
the tangles identified, and distinguished, are tangles of the set Sk of separations of V of order less than k (which may or may not include the qualities in S of order <k themselves), giving rise to clusters of separations (CloSs) of increasing order that become more and more specific as k grows;
a first set T is a separation of V, which are efficient in that T contains, for any two CloSs, a separation of lowest possible order that distinguishes these two CloSs in that one of the two CloSs contains this separation but the other of the two CloSs does not;
a second set T now witnesses that a set Sk of separations of order less than a fixed k has no CloS.

3. The method according to claim 1, wherein a list of potential qualities comprises a set of qualities each determined by a respective range for a corresponding physical and/or medical parameter,
and wherein testing the elements of V comprises, for each element v of V and for each quality q contained in said set,
measuring, with regard to v, the parameter respectively corresponding to q, and
determining whether the measured parameter lies in the respective range determining q.

4. A method of detecting mindsets each defined by respective properties and/or opinions shared by a respective plurality of persons of a population, the method comprising:
performing the method of claim 1, wherein the set V is a representative subset of a population and wherein the potential qualities contained in the list S each represent a possible property and/or opinion a human may have, and
defining the mindsets to be the identified CloQs or CloSs.

5. A method of appointing a representative body, the method comprising:
detecting a plurality of mindsets in a population by performing the method according to claim 4, and
for at least one determined mindset delegating a person whose opinions match best with the at least one determined mindset.

6. A method of discovering combinations of medical symptoms, which in combination constitute an illness, the method comprising:
performing the method according to claim 1, wherein the set V is a set of patients and wherein the potential qualities contained in the list S each define a symptom a patient may have, and
defining the combinations to be the identified CloQs or CloSs.

7. A method of defining teaching modes, the method comprising:
performing the method according to claim 1, wherein V is a set of students, and wherein the potential qualities contained in the list S each represent a respective teaching technique, a student in V being considered to have such quality if the respective teaching technique has been appraised to be advantageous for the student, and
defining the teaching modes to be the identified CloQs or CloSs.

8. A method for providing, responsive to a query of a respective word, a meaning thereof in terms of a set of properties, the method comprising:
performing the method according to claim 1, wherein the set V is a collection of words, and wherein the potential qualities contained in the list S each relate to a respective property, a word in V being considered to have such property if at least one item represented by the word has the property,
determining at least one of the identified CloQs or CloSs which the respectively queried word matches best, and
providing the determined at least one CloQ or CloS as the meaning of the queried word.

9. A method of grouping goods in a physical or online store, the method comprising:
performing the method according to claim 1, wherein the set V is a set of customers of a shop, and wherein the potential qualities each are related to a respective item sold in the shop, wherein a customer in V is considered to have such quality if he has bought the item related to the quality in the shop, and
arranging the goods in the store in one or more groups based on one or more of the identified CloQs or CloSs and/or running at least one advertising campaign based on the one or more of the identified CloQs or CloSs.

10. A method of grouping clients with respect to their purchase behavior, the method comprising:
performing the method according to claim 1, wherein the elements of the set V are items offered in a shop, and wherein the potential qualities each are related to a respective customer of the shop, wherein an item in V is considered to have such quality if it has been purchased in the shop by the customer related to the quality,
determining at least one group of the clients based on at least one of the identified CloQs or CloSs.

11. A method of developing drugs, the method comprising:
performing the method according to claim 1, wherein the set V is a set of pathogens, and wherein the potential qualities contained in the list S each relate to a respective pathogen feature; and
determining, for at least one of the identified CloQs or CloSs, a drug targeting the qualities included in the identified CloQ or CloS, respectively.

12. A method of automatically providing a class of a queried text, the method comprising:
performing the method according to claim 1, wherein the set V is a set of sample texts, and the potential qualities contained in the list S each relate to a respective text characteristic;
assigning the identified CloQs or CloSs to respective classes;
determining at least one of the identified CloQs or CloSs which the respectively queried text matches best; and
providing the class assigned to the best-matching CloQ or CloS of the queried text.

13. A method of sequencing DNA and/or proteins, the method comprising:
performing the method according to claim 1, wherein the set V is a set of DNA or protein molecules, and wherein the potential qualities contained in the list S each refer to an occurrence of specified nucleotides or amino acids, or combinations thereof, at given positions in the DNA or protein molecules, and
classifying the DNA or protein molecules corresponding to these nucleotides or amino acid sequences by the CloQs or CloSs identified.

14. A system for artificial exploitation of a data set containing relations of objects each with at least one respective quality, the objects each being an element of a given set V, the qualities each being included in a given list S of potential qualities, wherein the system comprises at least one computer configured
to search for subsets of S as CloQs: as groups of qualities from S that often occur together in the objects of V and form a tangle of S; and
if such CloQs exist for the data set, to compute a nested set T of at most |V| Boolean expressions of qualities from S that suffice to distinguish the CloQs, in that for any two CloQs there exists a combination tϵT of qualities which is shared by the qualities in one of the two CloQs but not by those in the other of the two CloQs.

15. The system according to claim 14, wherein the at least one computer is further configured to compute, if no CloQ exists for the data set, a nested set τ of at most |V| Boolean expressions of qualities from S which, between them, witness that no CloQ exists in S.

16. The system according to claim 14, wherein the system further comprises at least one measuring device configured to measure a physical and/or medical parameter and to transmit the measured parameter to the at least one computer.

17. A non-transitory computer readable storage means comprising instructions stored thereon which, when processed by a computer, carry out the method according to claim 1.

18. The method of claim 12, wherein the respective text characteristic comprises syntax and/or a number of appearances of a predetermined word in a respective text.

19. The method of claim 12, wherein the respective classes comprise a topic, a genre, and/or an author.
